(19) United States
(12) Patent Application Publication
(10) Pub. No.: US 2003/0032065 A1
(43) Pub. Date: Feb. 13, 2003

(54) Ensemble-based strategy for the design of protein pharmaceuticals

(76) Inventors: Vince Hilser, Galveston, TX (US); Robert O. Fox, Galveston, TX (US)

Correspondence Address: FULBRIGHT & JAWORSKI, LLP, 1301 MCKINNEY, SUITE 5100, HOUSTON, TX, 77010-3095, US

(21) Appl. No.: 10/096,177

(22) Filed: Mar. 12, 2002

Related U.S. Application Data
(60) Provisional application No. 60/275,259, filed on Mar. 12, 2001.

(51) Int. Cl. G01N 33/53; G06G 7/48; G06G 7/58; G06F 19/00; C12P 21/02

(52) U.S. Cl. 435/7.1; 435/69.1; 703/11; 702/19

(57) ABSTRACT

The present invention provides a method to generate and analyze ensembles of peptide and protein conformers and design proteins to exhibit desired characteristics. The present invention is particularly useful in protein pharmaceutical design.

This application claims priority to U.S. Provisional Application No. 60/275,259, which was filed on Mar. 12, 2001.

The work herein was supported by grants from the United States Government. The United States Government may have certain rights in the invention.

### BACKGROUND OF THE INVENTION

I. Field of the Invention

The invention generally relates to the field of structural biology and protein modeling, specifically computer-assisted methods of optimizing various characteristics of a protein. The invention is particularly useful in the design of protein pharmaceuticals.

II. Related Art

The three-dimensional structure of proteins has been determined in a number of ways. Perhaps the best known way of determining protein structure involves the use of the technique of x-ray crystallography. Using this technique, it is possible to elucidate the three-dimensional structure with good precision. Additionally, protein structure may be determined through the use of the techniques of neutron diffraction, or by nuclear magnetic resonance

The three-dimensional structure of many proteins may be characterized as having internal surfaces (directed away from the aqueous environment in which the protein is normally found) and external surfaces (which are exposed to the aqueous environment). Through the study of many natural proteins, researchers have discovered that hydrophobic residues (such as tryptophan, phenylalanine, leucine, isoleucine, valine, or methionine) are most frequently found on the internal surface of protein molecules. In contrast, hydrophilic residues (such as aspartate, asparagine, glutamate, glutamine, lysine, arginine, serine, and threonine) are most frequently found on the external protein surfaces. The amino acids alanine, cysteine, glycine, histidine, proline, serine, tyrosine, and threonine are encountered with more nearly equal frequency on both the internal and external protein surfaces.

The biological properties of proteins depend directly on the protein's three-dimensional (3D) conformation. The conformation determines the activity of enzymes, the capacity and specificity of binding proteins, and the structural attributes of receptor molecules. Each protein has an astronomical number of possible conformations (about 10¹⁶ for a small protein of 100 residues, and there has been no reliable method for picking the one conformation that predominates in aqueous solution. A second difficulty is that there are no accurate and reliable force laws for the interaction of one part of a protein with another part and with water. These and other factors have contributed to the enormous complexity of determining the most probable relative location of each residue in a known protein sequence.

The protein folding problem, the problem of determining a protein's three-dimensional tertiary structure from it's amino acid sequence, was first formulated more than half a century ago. Early observations and later experiments have lead to the contemporary view that protein conformation is determined solely by the amino acid sequence and that there exists a unique native conformation in which residues distant in sequence but proximate in space engender a close-packed core enriched in hydrophobic residues. As a result of the revolution in molecular biology, the number of known protein sequences is about 50 times greater than the number of known three-dimensional protein structures. This disparity hinders progress in many areas of biochemistry because a protein sequence has little meaning outside the context of the three-dimensional structure.

Structure-based drug design uses the three-dimensional structure of protein-drug complexes to predict more active compounds e.g., compounds with better drug properties and similar potency. The key is that structure, motion and energies of proteins and their inhibitors are the cause of activities of drugs.

Drug design has historically involved “discovering” a particular chemical substance that interacts in some way with receptors, e.g., proteins in the living cells of a mammalian body. As proteins are made up of polypeptides, it is not surprising that some effective drugs are also peptides, or are patterned after peptides. Generally, for two peptides to effectively interact with each other, e.g., one as a protein receptor and the other as a drug, it is necessary that the complex three-dimensional shape (“conformation”) of one peptide assume a compatible conformation that allows the two peptides to fit and bind together in a way that produces the desired result. For example, the complex shape or conformation of a first peptide has been compared to a “lock”, and the corresponding requisite shape or conformation of the receptor as a “key” that unlocks (i.e., produces the desired result within) the first peptide. This “lock-and-key” analogy emphasizes that only a properly conformed key (second peptide or compound patterned thereafter) is able to fit within the lock (first peptide) in order to “unlock” it (produce a desired result). Further, even if the key fits in the lock, it must have the proper composition in order for it to perform its function. That is, the second peptide must contain the right elements in the right spatial arrangement and position in order to properly bind with the first peptide, e.g., receptor protein. Discovering or predicting the proper conformation or shape of the key, or second peptide or compound patterned thereafter, is thus a major objective of any drug design.

Another objective in drug design is designing a drug with few intrinsic or extrinsic properties that elicit an immune response. It is known that proteins elicit an immune response. Proteins have the ability to engage T cells, which contributes to inducing most antibody responses and are required for immunological memory. The T cells recognize antigens as peptide fragments of proteins bound to major histocompatibility complex molecules (MHC). Certain properties of proteins have a greater influence on the immunogenicity of the protein. For example, the larger and more complex a protein, and the more distant its relationship to self proteins, the more likely it is to elicit a response. Thus, the larger and more distinct a protein antigen, the more likely it is to contain peptides that are recognized by the T cells. Also, particulate or aggregated antigens are more immunogenic because they are taken up more efficiently by cells, e.g., antigen-presenting cells.

The protein modeling approach of the present invention provides a method of predicting the possible structures of a peptide or protein. Examples of uses of the present invention are to increase solubility or decrease aggregation or immunogenicity of the peptide or protein. This method allows the prediction of structures for both the stable and unstable regions of a peptide or protein.

### SUMMARY OF THE INVENTION

The present invention provides a method to generate and analyze ensembles of peptide and protein conformers and design proteins to exhibit desired characteristics. The present invention is particularly useful in protein pharmaceutical design.

An embodiment of the present invention is a method of designing a protein pharmaceutical exhibiting optimized pharmaceutical properties comprising the steps of: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide optimized pharmaceutical properties.

Another embodiment of the present invention is a method of designing a protein pharmaceutical exhibiting optimized pharmaceutical properties comprising the steps of: inputting a high resolution structure of said protein pharmaceutical into a computer-assisted modeling program; measuring pharmaceutical delivery properties of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting optimized pharmaceutical properties; mutating the amino acid sequence of the protein pharmaceutical to provide a variant; repeating the above steps for a given number of cycles in order to prepare a library of ensemble-derived properties for each variant; deriving a parametric equation using the pharmaceutical properties of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has optimized pharmaceutical properties over the prior lead tested; comparing the variant to the goal for each property; and creating the variant of the protein pharmaceutical with the structural characteristics found by the above steps to provide optimized pharmaceutical properties. In specific embodiments, the method comprises repeating steps of obtaining a large number of variants based on the constraint set; testing variants to select a lead mutation set based on a database; testing the lead in the parametric equation; and determining if the lead has optimized pharmaceutical properties over the prior lead tested until the pharmaceutical properties have been sufficiently optimized for use.

Another specific embodiment is a method of designing a protein pharmaceutical exhibiting increased binding affinity between the protein pharmaceutical and a ligand comprising the steps of: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide increased binding affinity between the protein pharmaceutical and a ligand.

An additional embodiment of the present invention is a method of designing a protein pharmaceutical exhibiting increased binding affinity between the protein pharmaceutical and a ligand comprising the steps of: inputting a high resolution structure of said protein pharmaceutical computer-assisted modeling program; determining binding affinity between the protein pharmaceutical and the ligand; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein capable of binding the ligand; mutating the amino acid sequence of the protein pharmaceutical to provide a variant; repeating the steps above for a given number of cycles in order to prepare a library of ensemble-derived properties for each variant; deriving a parametric equation using the binding affinities of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has an increased binding affinity over the prior lead tested; and creating the variant of the protein pharmaceutical with the structural characteristics found by the above steps to exhibit increased binding affinity to the ligand. In specific embodiments, the method further comprises repeating steps obtaining a large number of variants based on the constraint set; testing variants to select a lead mutation set based on a database; testing the lead in the parametric equation; and determining if the lead has an increased binding affinity over the prior lead tested until the binding affinity has been sufficiently optimized for use as a protein pharmaceutical.

In specific embodiments, the binding affinity is determined by surface plasmon resonance. Yet further, the ligand is a protein or non-protein. More particularly, the protein inhibits the binding of a ligand to a receptor by binding the non-protein ligand at the receptor-binding site.

In further specific embodiments, determining the fraction of conformations capable of binding and the binding affinity comprises performing van der Waals calculations with the protein and the ligand to verify whether the conformation is sterically allowed.

In another specific embodiment, determining the fraction of conformations capable of binding and the binding affinity comprises determining the association or dissociation constant of the binding between the protein and the ligand.

In specific embodiments, the method further comprises determining the conformers that decrease the entropy of binding by stabilizing structures similar to that of the protein in a bound state with the ligand.

Another embodiment of the present invention is a method of designing a protein pharmaceutical exhibiting decreased aggregation comprising the steps of: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide decreased aggregation.

Another embodiment is a method of designing a protein pharmaceutical exhibiting decreased aggregation comprising the steps of: inputting a high resolution structure of said protein pharmaceutical computer-assisted modeling program; measuring aggregation of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting decreased aggregation; mutating the amino acid sequence of the protein pharmaceutical to provide a variant; repeating steps above for a given number of cycles in order to prepare a library of ensemble-derived properties for each variant; deriving a parametric equation using the aggregation values of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has an decreased aggregation over the prior lead tested; and creating the variant of the protein pharmaceutical with the structural characteristics found by the above steps to exhibit decreased aggregation.

In specific embodiments, the method further comprises repeating the steps of obtaining a large number of variants based on the constraint set; testing variants to select a lead mutation set based on a database; testing the lead in the parametric equation; and determining if the lead has an decreased aggregation over the prior lead tested until the aggregation has been sufficiently optimized for use as a protein pharmaceutical. Specifically, aggregation is measured by light scattering at 360 nm. Yet further, the number of hydrophobic residues exposed on the surface of the protein is reduced. Specifically, the number of unfolded regions found in equilibrium is reduced. Yet further, in specific embodiments, the number of glutamine/asparagine-rich domains is decreased.

Another embodiment of the present invention is a method of designing a protein pharmaceutical exhibiting increased solubility comprising the steps of: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide increased solubility.

Yet further, another embodiment of the present invention is a method of designing protein pharmaceuticals exhibiting increased solubility comprising the steps of: inputting a high resolution structure of said protein pharmaceutical computer-assisted modeling program; measuring solubility of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting increased solubility; mutating the amino acid sequence of the protein pharmaceutical to provide a variant; repeating the steps above for a given number of cycles in order to prepare a library of ensemble-derived properties for each variant; deriving a parametric equation using the solubility values of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has an increased solubility over the prior lead tested; and creating the variant of the protein pharmaceutical with the structural characteristics found by the above steps to exhibit increase solubility.

In specific embodiments, the method further comprises repeating the steps of obtaining a large number of variants based on the constraint set; testing variants to select a lead mutation set based on a database; testing the lead in the parametric equation; and determining if the lead has an increased solubility over the prior lead tested until the solubility has been sufficiently optimized for use as a protein pharmaceutical. In further specific embodiments, solubility is measured by determining the free transfer energy of the protein pharmaceutical.

In further specific embodiments, the number of polar residues on the surface of the protein is increased. Yet further, the number of nonpolar residues on the surface of the protein is decreased. Also, the net charge of the protein is increased.

Another embodiment of the present invention is a method of designing a protein pharmaceutical exhibiting decreased immunogenic effects comprising the steps of: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide decreased immunogenic effects.

Another specific embodiment is a method of designing a protein pharmaceutical exhibiting decreased immunogenic effects comprising the steps of: inputting a high resolution structure of said protein pharmaceutical computer-assisted modeling program; measuring immunogenic effects of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting decreased immunogenic effects; mutating the amino acid sequence of the protein pharmaceutical to provide a variant; repeating the steps above for a given number of cycles in order to prepare a library of ensemble-derived properties for each variant; deriving a parametric equation using the immunogenic effect values of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has decreased immunogenic effects over the prior lead tested; and creating the variant of the protein pharmaceutical with the structural characteristics found by the above steps to exhibit decreased immunogenic effects.

In specific embodiments, the method further comprises repeating the steps of obtaining a large number of variants based on the constraint set; testing variants to select a lead mutation set based on a database; testing the lead in the parametric equation; and determining if the lead has decreased immunogenic effects over the prior lead tested until the immunogenic effects have been sufficiently optimized for use in a protein pharmaceutical. Specifically, the immunogenic effects are determined by ELISA.

In further embodiments, the protein pharmaceutical exhibits a decreased tendency to aggregate with other molecules of the protein pharmaceutical. More specifically, the protein pharmaceutical exhibits a decreased tendency to bind to the proteins of the major histocompatability complex. In specific embodiments, the protein pharmaceutical is a protein of more than 5,000 Daltons. Yet further, the protein pharmaceutical is resistant to processing by the endosomal pathway.

A further embodiment of the present invention is a protein pharmaceutical exhibiting optimized pharmaceutical properties having structural characteristics determined by a method comprising the steps of: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide optimized pharmaceutical properties. In specific embodiments, the protein pharmaceutical is systemically or mucosally administered to a subject in a therapeutically effective amount. One of skill in the art realizes that a subject comprises a human or a non-human animal.

Another embodiment of the present invention is a protein pharmaceutical exhibiting optimized pharmaceutical properties having structural characteristics determined by a method comprising the steps of: inputting a high resolution structure of said protein pharmaceutical computer-assisted modeling program; measuring pharmaceutical properties of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting optimized pharmaceutical properties; mutating the amino acid sequence of the protein pharmaceutical to provide a variant; repeating steps above for a given number of cycles in order to prepare a library of ensemble-derived properties for each variant; deriving a parametric equation using the pharmaceutical properties of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has optimized pharmaceutical properties over the prior lead tested; comparing the variant to the goal for each property; and creating the variant of the protein pharmaceutical with the structural characteristics found by the steps above to provide optimized pharmaceutical properties. Specifically, the protein pharmaceutical is optimized by repeating the steps obtaining a large number of variants based on the constraint set; testing variants to select a lead mutation set based on a database; testing the lead in the parametric equation; and determining if the lead has optimized pharmaceutical properties over the prior lead tested; until the pharmaceutical properties have been sufficiently optimized for use.

A further embodiment is a protein pharmaceutical exhibiting increased binding affinity between the protein and a ligand, having the structural characteristics determined by a method wherein the steps comprise: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide increased binding affinity between the protein pharmaceutical and a ligand.

Another embodiment of the present invention is a protein pharmaceutical exhibiting increased binding affinity between said protein and a ligand, having the structural characteristics determined by a method wherein the steps comprise: inputting a high resolution structure of said protein pharmaceutical computer-assisted modeling program; determining binding affinity between the protein pharmaceutical and the ligand; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein capable of binding the ligand; mutating the amino acid sequence of the protein pharmaceutical to provide a variant; repeating the steps above for a given number of cycles in order to prepare a library of ensemble-derived properties for each variant; deriving a parametric equation using the binding affinities of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has an increased binding affinity over the prior lead tested; and creating the variant of the protein pharmaceutical with the structural characteristics found by the above steps to exhibit increased binding affinity to the ligand. Yet further, the protein pharmaceutical can be optimized by repeating the steps of obtaining a large number of variants based on the constraint set; testing variants to select a lead mutation set based on a database; testing the lead in the parametric equation; and determining if the lead has an increased binding affinity over the prior lead tested until the binding affinity has been sufficiently optimized for use.

Another embodiment is a protein pharmaceutical exhibiting decreased aggregation, having the structural characteristics determined by a method wherein the steps comprise: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide decreased aggregation.

Another specific embodiment of the present invention is a protein pharmaceutical exhibiting decreased aggregation in comparison to an initial structure having structural characteristics determined by a method comprising the steps of: inputting a high resolution structure of said protein pharmaceutical computer-assisted modeling program; measuring aggregation of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting decreased aggregation; mutating the amino acid sequence of the protein pharmaceutical to provide a variant; repeating the steps above for a given number of cycles in order to prepare a library of ensemble-derived properties for each variant; deriving a parametric equation using the aggregation values of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has an decreased aggregation over the prior lead tested; and creating the variant of the protein pharmaceutical with the structural characteristics found by the above steps to exhibit decreased aggregation. Specifically, the protein pharmaceutical can be optimized by repeating steps obtaining a large number of variants based on the constraint set; testing variants to select a lead mutation set based on a database; testing the lead in the parametric equation; and determining if the lead has an decreased aggregation over the prior lead tested until the aggregation has been sufficiently optimized for use.

A further embodiment of the present invention is a protein pharmaceutical exhibiting increased solubility, having the structural characteristics determined by a method wherein the steps comprise: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide increased solubility.

Another embodiment is a protein pharmaceutical exhibiting increased solubility in comparison to an initial structure having structural characteristics determined by a method comprising the steps of: inputting a high resolution structure of said protein pharmaceutical computer-assisted modeling program; measuring solubility of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting increased solubility; mutating the amino acid sequence of the protein pharmaceutical to provide a variant; repeating steps above for a given number of cycles in order to prepare a library of ensemble-derived properties for each variant; deriving a parametric equation using the solubility values of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has an increased solubility over the prior lead tested; and creating the variant of the protein pharmaceutical with the structural characteristics found by the above steps to exhibit increase solubility. In specific embodiments, the protein pharmaceutical can be optimized by repeating the steps of obtaining a large number of variants based on the constraint set; testing variants to select a lead mutation set based on a database; testing the lead in the parametric equation; determining if the lead has an increased solubility over the prior lead tested until the solubility has been optimized for use.

Another embodiment of the present invention is a protein pharmaceutical exhibiting decreased immunogenic effects, having the structural characteristics determined by a method wherein the steps comprise: obtaining a test data set of variants of the protein pharmaceutical; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical with the structural characteristics found by the above steps to provide decreased immunogenic effects.

A further embodiment is a protein pharmaceutical exhibiting decreased immunogenic effects in comparison to an initial structure having structural characteristics determined by a method wherein the steps comprise: inputting a high resolution structure of said protein pharmaceutical computer-assisted modeling program; measuring immunogenic effects of the protein pharmaceutical; obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; determining a fraction of conformations of the protein exhibiting decreased immunogenic effects; mutating the amino acid sequence of the protein pharmaceutical to provide a variant; repeating steps above for a given number of cycles in order to prepare a library of ensemble-derived properties for each variant; deriving a parametric equation using the immunogenic effect values of each variant and the library of ensemble-derived properties; identifying an initial lead variant; initializing a constraint set; obtaining a large number of variants based on the constraint set; testing variants to select a lead mutation set based on a database; testing the lead in the parametric equation; determining if the lead has decreased immunogenic effects over the prior lead tested; and creating the variant of the protein pharmaceutical with the structural characteristics found by the above steps to exhibit decreased immunogenic effects. Specifically, the protein pharmaceutical can be optimized by repeating the steps of obtaining a large number of variants based on the constraint set; testing variants to select a lead mutation set based on a database; testing the lead in the parametric equation; and determining if the lead has decreased immunogenic effects over the prior lead tested until the immunogenic effects have been optimized for use.

A further aspect of the invention is a computer system that can implement the described methods. The computer system has a software program coded to perform the described methods. Preferably, a software program would read protein data from a database or from an input file. One embodiment of such a computer system for designing a protein pharmaceutical exhibiting optimized pharmaceutical is a database containing a test data set of variants of a protein pharmaceutical; and a software program coupled with the database. The software program would be coded or adpated to execute instructions for preparing a library of ensemble derived properties for the test data set, generating experimental data for a given property for each protein variant within the test data set, deriving a parametric equation using the experimental data and the library of ensemble derived properties, and creating a protein pharmaceutical structure with structural characteristics found by the above steps. Addtionally, the software would be able to determine one or more of the following with respect to the designed pharmaceutical: provide optimized pharmaceutical properties, provide increased binding affinity between the protein pharmaceutical and a ligand, and provide decreased immunogenic effects.

Another aspect of the invention is a computer-readable storage medium having stored therein a software program that is capable of executing the methods described herein. The comptuer-readable medium may be any storage-readable medium utilized by a computer, for purposes of illustration but not for limitation, may include floppy disks, hard drives, storage drives, disk packs, ROM, RAM, PC cards, optical media, and magnetic media. In one embodiment, such a computer-readable storage medium has a software program that executes the steps of preparing a library of ensemble derived properties from a test data set of variants of a protein pharmaceutical, generating experimental data for a given property for each protein variant within the test data set, and deriving a parametric equation using the experimental data and the library of ensemble derived properties, and creating a protein pharmaceutical structure with the structural characteristics found by the aforementioned steps. Morever, the software program would be able to determine one or more of the following with respect to the designed pharmaceutical: provide optimized pharmaceutical properties, provide increased binding affinity between the protein pharmaceutical and a ligand, and provide decreased immunogenic effects

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF SUMMARY OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Flow chart of BEST/MPMOD program as an analysis tool.

FIG. 2A and FIG. 2B. Flow chart of BEST/MPMOD program as a predictive tool to be used in the development of protein pharmaceuticals with optimized characteristics.

FIG. 3. Illustration of the COREX algorithm for generating artificially folded states. For this example, a total of 12 different partitionings have been generated using a block of windows of 12 residues each.

FIG. 4. Natural logarithm (bars) of the calculated and experimental protection factors for λ6-85. The calculated values were determined as described by Hilser and Freire 1996. The solid line above the calculated values represents the residue stability constant as defined. This quantity is defined for all residues independent of whether they exhibit protection or not. Shown also in the figure are the corresponding elements of secondary structure. The good agreement between calculated and experimental values indicates that the calculated ensemble captures the general features of the actual ensemble and that the network of cooperative interactions in the protein are represented accurately in this model.

FIG. 5(A). Two views of the X-ray structure of Src-homology-3 (SH3) domain of the C. elegans protein SEM5 in complex with the peptide Sos. (B) Residue folding constants for SEM5. Gray bars indicate residues in loop regions which show low stability (colored gray in (A)). (C) Schematic representation of the SEM5 ensemble showing the binding-competent sub ensemble in the absence (I.) and presence (II.) of peptide.

FIG. 6. Flow chart of the MPMOD program.

FIG. 7. Disulfide-bonded random conformations for the ensemble CCHPQCGMVEEC. Each conformer has two cross-linked disulfide bonds. The randomly generated conformer has various conformations.

FIG. 8. Interactions for the peptide-streptavidin complex. Here the peptide has two disulfide bonds that are cross-linked. The HPQ motif is sitting in the binding pocket and there are three hydrogen bonds involving in the interaction for the complex.

FIG. 9. The number of chances for each residue of the peptide CCHPQCGMVEEC to collide with the target streptavidin.

FIG. 10A and FIG. 10B. Flow chart of the MPMOD program (Fast Mod).

FIG. 11A and FIG. 11B. Flow chart of the MPMOD program (Slow Mod).

FIG. 12. Flow chart of the MPMOD program (Loop Generation).

FIG. 13. Flow chart of the MPMOD program's modeling of disulfide bonds.

FIG. 14. Flow chart for the binding test.

### DETAILED DESCRIPTION OF THE INVENTION

The present application includes methods of modifying a peptide or protein in order to optimize various characteristics by using the combination of multiple computer-assisted methods of protein modeling. The invention is particularly useful in the design of protein pharmaceuticals.

I. Definitions

“A” or “an”, as used herein the specification, may mean one or more. As used herein in the claim(s), when used in conjunction with the word “comprising”, the words “a” or “an” may mean one or more than one.

Aggregation, as used herein, refers to the interaction of proteins, usually non-specific, to form a complex that may or may not be covalently linked.

Another, as used herein, may mean at least a second or more.

Autologous protein, polypeptide or peptide, as used herein, refers to a protein, polypeptide or peptide which is derived or obtained from an organism.

Based upon a tertiary structure, as used herein, refers to a structure that possesses a similar backbone structure to that of the original structure that it is referred to being based upon.

Configuration, as used herein, refers to different conformations of a protein molecule that have the same chirality of atoms.

Conformation or conformer, as used herein, refers to various nonsuperimposable three-dimensional arrangements of atoms that are interconvertible without breaking covalent bonds.

Computer modeling, as used herein, refers to the construction of patterns using raw data to simulate an object or the interaction of objects using a computer. For example, computer modeling is used to determine the size, shape, and interaction of certain compounds in order to develop treatments associated to a specific disease.

Computer simulation, as used herein, refers to a software program that runs on any size computer that attempts to simulate some phenomenon based on a scientist's conceptual and mathematical understanding of the phenomenon. The scientist's conceptual understanding is reduced to an algorithmic or mathematical logic, which is then programmed in one of many programming languages and compiled to produce a binary code that runs on a computer. Also, the act of running such a code on a computer.

Constrained, as used herein, refers to a limitation in the conformational space that the peptide may adopt.

Database, as used herein, refers to any compilation of information regarding the relation of experimental and analytical data of a protein. The database used may be publicly available, commercially available or one created by the inventors.

Disulfide bridge or disulfide bond, as used herein, refers to a covalent bond between the sulfur atoms of two cysteines.

Generate or generating, as used herein, refers the act of defining or originating by the use of one or more operations. Skilled artisans using the invention may create the matter or data themselves or locate the matter or data elsewhere and utilize it in the practice of the invention. One skilled in the art realizes that in this invention all of the test data or experimental data may be obtained commercially or publicly or generated by procedures and techniques defined herein. The terms “generating” and “obtaining” are mutally inclusive as used herein.

Immunogenicity, as used herein, refers to the amount of an immune response that is produced by the protein, specifically the amount of binding to proteins of the major histocompatibility complex (MHC) by the protein in question.

Lead, as used herein, refers to the first or foremost position or precedent. Thus, the lead variant, is the first or initial variant that may be tested. The lead may be not be the final variant, it is merely a starting point.

Ligand, as used herein, refers to a proteinaceous or non-proteinaceous compound. The ligand may be, but is not limited to, a receptor, an enzyme, a coenzyme, or a non-proteinaceous chemical compound.

Loop, as used herein, are turns in the polypeptide chain that reverse the direction of the polypeptide chain at the surface of the molecule.

Mutation(s), as used herein, refers to a change of one or more amino acids in a protein.

Parametric equation, as used herein, refers to an equation containing variables related to the populated conformational states for a given variant. The equation utilizes the experimental data acquired for each variant and the library of ensemble-derived properties.

Peptide, as used herein, refers to a chain of amino acids with a defined sequence whose physical properties are those expected from the sum of its amino acid residues and there is no fixed three-dimensional structure).

Pharmaceutical properties, as used herein, refer to, but are not limited to, binding affinity, aggregation, solubility, and immunogenic effects.

Protein, as used herein, refers to a chain of amino acid residues usually of defined sequence, length and three dimensional structure. The polymerization reaction which produces a protein results in the loss of one molecule of water from each amino acid. Proteins are often said to be composed of amino acid residues. Natural protein molecules may contain as many as 20 different types of amino acid residues, each of which contains a distinctive side chain. A protein may be composed of multiple peptides.

Rotamer, as used herein refers to a low energy amino acid side chain information.

Solubility, as used herein, refers to the amount of the protein that can be dissolved in a given volume of a solvent.

Structural Characteristics, as used herein, refers to the characteristics that are determined using the computer-assisted program, such as, but not limited to folding characteristics, disulfide bonding, binding affinity, aggregation, solubility, immunogenicity, stablility, etc. Thus, one of skill in the art realizes that the present invention is used to determine any structural characteristic of a protein and this characteristic may be enhanced or reduced depending upon the application of use.

Template molecule, as used herein, refers to the protein to which the modified protein is binding.

Variant, as used herein, refers to a protein with a given set of mutation(s).

II. COREX Computer Modeling Strategy

The COREX algorithm estimates the entire set of statistical descriptors of the equilibrium folding pathway from the high-resolution structure of a protein, this set of values is used to predict global and residue-specific experimental observables, e.g., protein stability or hydrogen exchange protection factors. The three-dimensional structure is used as a template to generate a large ensemble of partially folded states, and the relative free energy of each state in the ensemble is calculated. Once the free energy of each state is estimated, the pattern function and remaining thermodynamic quantities are evaluated.

A. Generation of Partly Folded States by COREX Algorithm

The partition function

$$( Q = {\sum }_{i = 0}^{N} exp ( - Δ {G}_{i} / RT ) )$$

is a sum over all states of the protein. This is an astronomical number even for a small protein. For example, if each amino acid of a 100 residue protein had ten possible conformations, the total number of states would be 10¹⁰⁰, which is a computationally intractable number. Fortunately, protein folding is a highly cooperative process and most states have almost zero probability and do not contribute to the partition function. This fact permits a significant simplification of the enumeration problem. It is desirable to develop a set of selection rules that will allow the creation of a subset that only contains the states that contribute to the partition function. Since experimental properties can be predicted from the partition function, the accuracy of the resulting predictions are used to assess the validity of the assumptions used in the selection rules and to refine them.

An approach to generating an ensemble of intermediate states for a particular protein is to use the high-resolution structure of the native state as a template and in a systematic way use the computer to unfold predetermined regions of the molecule in all possible combinations. The resolution of the results depends on the size and number of the regions (called folding units) used to generate the partially folded states. There are two basic assumptions in this algorithm: (1) the folded regions in partially folded states are native-like; and (2) the unfolded regions are assumed to be devoid of structure. This approach predicts the general features of experimentally observed intermediates (Freire & Xie, 1994; Xie et al., 1994; Xie & Freire, 1994a,b), suggesting that in most cases the population of non-native intermediates is negligible. This conclusion is supported also by the measured energetics of these structures (Freire, 1995; Griko et al., 1994, 1995; Haynie & Freire, 1993; Xie et al., 1994), by the observation that the molten globule state of several proteins preserve the native fold (Jennings & Wright, 1993; Peng & Kim, 1994; Schulman et al., 1995), and by the general observation that even very early kinetic intermediates already display native-like features (e.g., see Jacobs & Fox, 1994; Jones & Matthews, 1995; Matthews, 1993; Radford et al., 1992b; Sosnick et al., 1994).

The COREX algorithm employs a block of windows of N_{w} amino acid residues each, which is used to partition the protein into different folding units. Each protein partition consists of Nᵤ folding units, where Nᵤ is equal to the ceiling of Nᵣₑₛ/N_{w}. Nᵣₑₛ is the total number of residues and N_{w} is the number of amino acid residues per window. The first partitioning of the protein is defined by moving the block of windows over the entire sequence of the protein beginning with the first residue. If Nᵣₑₛ/N_{w} is not an integer, the number of residues in the last unit is set equal to the remainder. This partitioning results in (2^{N}^{_{U,i}}−2) partially folded intermediates generated by folding and unfolding the units in all possible combinations. Once the first partitioning is performed, a second partitioning is defined by sliding the block of windows one amino acid residue in the sequence. This process is continued until the entire sequence has been exhausted. The total number of distinct states generated by using this procedure is equal to 2+Σ(2^{N}^{_{U,i}}−2), where the sum runs over all partitions and N_{U,i} is the number of folding units in partition i.

In order to test the stability of the results, different window sizes were used. In all cases studied, the results were independent of window size within the range considered. Folding units may or may not coincide with well-defined elements of secondary structure, thus eliminating any a priori judgement about their integrity or preferential stability over states in which those elements are not formed or only partially formed. This procedure generates a complete range of local, partial and globally unfolded conformations ranging from the size of the window itself (5 to 12 residues) to the entire protein molecule.

B. Calculation of Gibbs Energies

The partitioning scheme for hen egg white lysozyme (HEWL) generates a total of 32,757 different states. In order to estimate the probability of each of these states (ΔGᵢ=ΔHᵢ−TΔSᵢ) it is necessary to evaluate the free energy of each and every one of them.

The free energy of each conformational state was calculated using the empirical parameterization of the free energy developed earlier (D'Aquino et al., 1996; Gomez & Freire, 1995; Gomez et al., 1995; Xie & Freire, 1994a,b). This parameterization has been derived from the analysis of protein data. Briefly, this procedure involves calculation of the relative heat capacity (ΔCₚ), enthalpy (ΔH) and entropy (ΔS) of each state at the desired temperature.

The heat capacity change is a weak function of temperature and has been parameterized in terms of changes in solvent-accessible surface areas (ΔASA), since it originates mainly from changes in hydration (Gomez & Freire, 1995; Gomez et al., 1995; Murphy et al., 1992):

ΔCₚ=ΔC_{p,ap}+ΔC_{p,pol}

ΔCₚ=a_{C}(T)ΔASAₐₚ+b_{C}(T)ΔASAₚₒₗ

where the coefficients a_{C}(T)=0.45+2.63×10⁻⁴(T−25)−4.2×10⁻⁵(T−25)² and b_{C}(T)−0.26+2.85×10⁻⁴(T−25)−4.31×10⁻⁵(T−25)². In the equation above, ΔASA changes are in Å² and the heat capacity in cal K⁻¹mol⁻¹. In general, for low temperature calculations (<80° C.) the temperature-independent coefficients are sufficient (Gomez & Freire, 1995; Gomez et al., 1995).

The bulk of the enthalpy change also scales in terms of ΔASA changes and at the reference temperature of 60° C. it can be written as:

ΔH_{gen}(60)=a_{H}(60)ΔASAₐₚ+b_{H}(60)ΔASAₚₒₗ

where a_{H}(60)=−8.44 and b_{H}(60)=31.4, and the enthalpy is in cal/mol (Xie & Freire, 1994a,b).

In the calculation of the entropy change, two primary contributions are included; one due to changes in solvation and the other due to changes in conformational degrees of freedom (ΔS=ΔSₛₒₗᵥ+ΔS_{conf}). The entropy of solvation can be written in terms of the heat capacity if the temperatures at which the apolar and polar hydration entropies are zero (T*_{S,ap} and T*_{S,pol}) are used as reference temperatures:

ΔSₛₒₗᵥ=ΔS_{solv,ap}+ΔS_{conf,pol}

ΔSₛₒₗᵥ=ΔC_{p,ap}ln(T/T*_{S,ap})+ΔC_{p,pol}(T/T*_{S,pol})

T*_{S,ap} has been known to be equal to 385.15 K for some time (Baldwin, 1986; Murphy & Freire, 1992) and T*_{S,pol} has recently been found to be close to 335.15 K (D'Aquino et al., 1996).

Conformational entropies are evaluated by explicitly considering the following three contributions for each amino acid: (1) ΔS_{bu→ex}, the entropy change associated with the transfer of a side-chain that is buried in the interior of the protein to its surface; (2) ΔS_{ex→u}, the entropy change gained by a surface-exposed side-chain when the peptide backbone unfolds; and (3) ΔS_{bb}, the entropy change gained by the backbone itself upon unfolding. The magnitude of these terms for each amino acid residue has been estimated by computational analysis of the probability of different conformers as a function of the dihedral and torsional angles (D'Aquino et al., 1996; Lee et al., 1994). The conformational entropy values for all amino acids reported by D'Aquino et al. (1996) are reproduced in Table 1. Additional entropic contributions due to the presence of disulfide bridges were estimated as described by Pace et al. (1988). On average, these contributions account for about 95% of the entropy change for complete unfolding of the protein. The remaining unaccounted contributions (primarily protonation effects) were estimated from the difference between predicted and experimental Gibbs energies for complete unfolding under the specific experimental conditions and distributed evenly among all residues.

TABLE 1
Conformational entropies for amino acids.

| | ΔS_{bu→ex} | ΔS_{ex→u} | ΔS_{bb} |
|---|---|---|---|
| Amino Acid | (cal/K mol) | (cal/K mol) | (cal/K mol) |
| Ala | 0.00 | 0.00 | 4.1 |
| Arg | 7.11 | −0.84 | 3.4 |
| Asn | 3.29 | 2.24 | 3.4 |
| Asp | 2.00 | 2.16 | 3.4 |
| Cys | 3.55 | 0.61 | 3.4 |
| Gln | 5.02 | 2.12 | 3.4 |
| Glu | 3.53 | 2.27 | 6.5 |
| Gly | 0.00 | 0.00 | 3.4 |
| His | 3.44 | 0.79 | 2.18 |
| Ile | 1.74 | 0.67 | 3.4 |
| Leu | 1.63 | 0.25 | 3.4 |
| Lys | 5.86 | 1.02 | 3.4 |
| Met | 4.55 | 0.58 | 3.4 |
| Phe | 1.40 | 2.89 | 3.4 |
| Ser | 3.68 | 0.55 | 3.4 |
| Thr | 3.31 | 0.48 | 3.4 |
| Trp | 2.74 | 1.15 | 3.4 |
| Tyr | 2.78 | 3.12 | 3.4 |
| Val | 0.12 | 1.29 | 2.18 |

Entropy values reproduced from D'Aquino et al. (1996). ΔS_{bu→ex} and ΔS_{ex→u} are from Lee et al. (1994). For the purpose of approximating ΔS_{bb}, the amino acids have been divided into four groups: no β carbon (Gly); β carbon only (Ala); β branching (Val and Ile); and γ carbon (remaining amino acids).

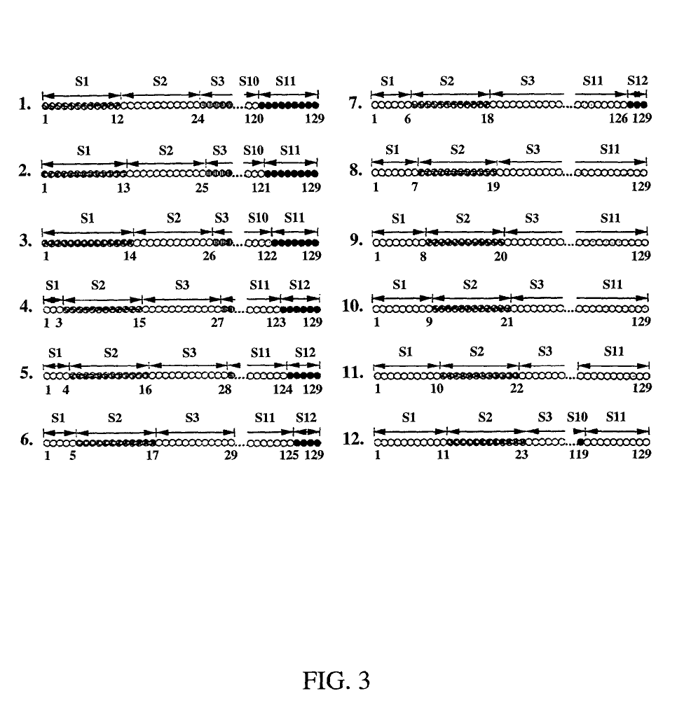

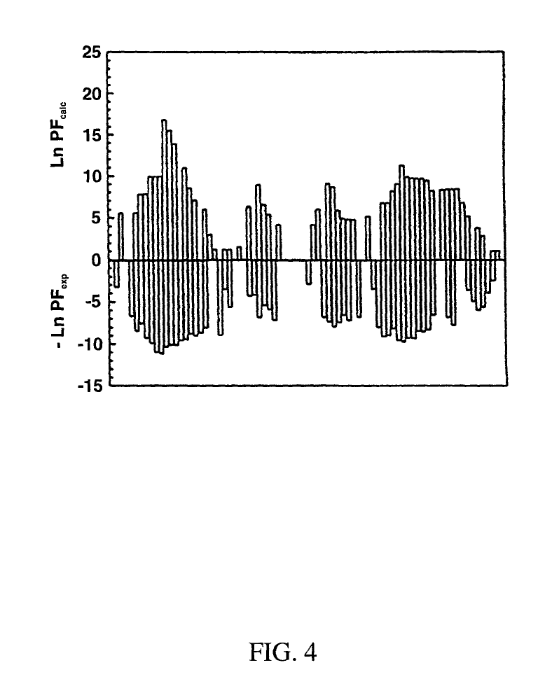

For each conformational state generated as described in FIG. 3, ΔASAₐₚ and ΔASAₚₒₗ are calculated using the Lee and Richards algorithm as described (Murphy et al., 1992). These ΔASA values are then used to calculate ΔH, ΔCₚ and ΔSₛₒₗᵥ values. In addition, for each residue in each conformational state, the state of the side-chain (buried, exposed in a folded region, exposed in an unfolded region) and the backbone (folded or unfolded) are determined in order to evaluate conformational entropies. Using this procedure, the free energies of all states generated for a given protein are evaluated.

C. Hydrogen Exchange Protection Factors

Under experimental conditions in which the so-called EX2 regime is obeyed, the equilibrium constant for the following reaction is measured by hydrogen exchange experiments (e.g., see Bai et al., 1995):

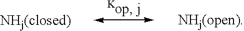

According to this reaction, K_{op,j} is equal to the ratio between the sum of the concentrations of all conformations in which residue j is open and therefore able to exchange protons with the solvent, and the sum of the concentrations of all conformations in which residue j is closed. The standard interpretation is that slowly exchanging protons exchange with the solvent only after becoming exposed to it as a result of local, partial or global unfolding. The residues that are unfolded in partially folded states are not the only residues that become exposed to the solvent. Also the residues located in the so-called complementary regions become exposed to the solvent, i.e., the residues located in portions of the protein that remain folded but were structurally complementary to the regions of the protein that became unfolded (Freire et al., 1993). The commonly reported hydrogen exchange protection factors, PFⱼ, are equal to the inverse of the K_{op,j} constants.

While the residue stability constants are purely thermodynamic quantities defined for all residues, the protection factors also contain non-thermodynamic contributions and are defined only for a subset of residues. Proline residues lack exchangeable amide protons and are not included. Residues with solvent-exposed amide groups in the native state are excluded (Pedersen et al., 1991). From a statistical standpoint, the protection factor for any given residue j can be defined as the ratio of the sum of the probabilities of the states in which residue j is closed, to the sum of the probabilities of the states in which residue j is open:

$${PF}_{j} = \frac{{\sum {P}_{i}}_{( stateswithresiduejclosed )}}{{\sum {P}_{i}}_{( stateswithresiduejopen )}} = \frac{{P}_{closed , j}}{{P}_{open , j}}$$

The statistical definition of the protection factors has the same form as that of the stability constants and can be expressed in terms of the folding probabilities as follows:

$${PF}_{j} = \frac{{P}_{f , j} - {P}_{f , xc , j}}{{P}_{nf , j} + {P}_{f , xc , j}}$$

the correction term P_{f,xc,j} is the sum of the probabilities of all states in which residue j is folded, yet exchange competent. It is evident that the hydrogen exchange protection factors PFⱼ are equal to the stability constants per residue, κ_{f,j} only when the P_{f,xc,j} terms are small. The most common situations in which a residue is folded but exposed to the solvent occurs when: (1) the amide group of the residue is exposed in the native state; and (2) the amide group of the residue becomes exposed due to its location in a region of the protein that is structurally complementary to an unfolded region. In the analysis presented here, prediction of the hydrogen exchange protection factors of the residues that exchange protons following the mechanism in equation (6) is done by calculation of the ensemble of P_{f,j} and P_{f,xcj} values. Of course, amide protons that exchange via different mechanisms (e.g., solvent penetration) will not be accounted for by this formalism.

It is clear from the above treatment that PFⱼ as well as κ_{f,j} are statistically defined quantities pertaining to an ensemble of conformations rather than to a chemical reaction between two discrete states.

III. Structural Distribution of Cooperative Interactions in Proteins

Cooperative interactions link the behavior of different amino acid residues within a protein molecule. As a result, the effects of chemical or physical perturbations to any given residue are propagated to other residues by an intricate network of interactions. Very often, amino acids “sense” the effects of perturbations occurring at very distant locations in the protein molecule. The inventors have investigated by computer simulation the structural distribution of those interactions. Cooperative interactions are not intrinsically bi-directional and different residues play different roles within the intricate network of interactions existing in a protein. The effect of a perturbation to residue j on residue k is not necessarily equal to the effect of the same perturbation to residue k on residue j. A computer algorithm aimed at mapping the network of cooperative interactions within a protein has been created by the inventors that exhaustively performs single site thermodynamic mutations to each residue in the protein and examines the effects of those mutations on the distribution of conformational states.

A. Mapping Cooperativity: SSTM.

Cooperativity can be examined by changing the free energy of all states in which a particular residue is folded, in essence performing a nonperturbing energy mutation of that residue. The resultant change in the statistical weight of all states in the numerator of

$${K}_{f , j} = \frac{\sum {P}_{f , j}}{\sum {P}_{nf , j}}$$

leads to a redistribution of the probabilities. As the subset of states in which a particular residue is folded (and unfolded) differs for each residue, the effect of a thermodynamic mutation will be specific for each residue in the protein. By performing individual thermodynamic mutations to each residue in the protein, it is possible to evaluate the effect of a change in each residue on all other residues. The end result of the SSTM analysis is a map from which the cooperative network of interactions within the protein can be deduced.

The directionality of cooperative behavior can be analyzed by considering three different effects: (i) cooperative response (i.e., the response of residue j to a mutation anywhere in the protein); (ii) donor cooperativity (i.e., the effect of a mutation in residue j on other residues); and (iii) mutual perturbation/response (i.e., the product of the effect of a mutation to residue j on k and the effect of a mutation to residue k on j, normalized by the magnitude of the perturbations;

$$Δ Δ {G}_{MPR} = - RT \cdot ln ( \frac{{κ}_{f , k}^{Mutk} \cdot {κ}_{f , j}^{Mutj}}{{κ}_{f , j}^{Mutk} \cdot {κ}_{f , k}^{Mutj}} )$$

where ΔΔG_{MPR} is defined as the mutual perturbation response free energy, κ_{f,j}^{Mutk} and κ_{f,k}^{Mutj} are the stability constants of residue j and k on mutation of residues k and j, and κ_{f,j}^{Mutj} and κ_{f,k}^{Mutk} are the stability constants of residues j and k on mutation of residues j and k, respectively).

B. Cooperative Response:

Mutations do not extend to all residues in the protein; however, there is a subset of residues that always are affected independently of the location of the perturbation. These residues are the most stable residues in the protein. The origin of this behavior can be explained by separating the contributions of partially folded states from those of the folded and unfolded states:

$${κ}_{f , j} = \frac{{P}_{N} + \sum {P}_{f , j}}{{P}_{U} + \sum {P}_{n f , j}} ,$$

where the summation in the numerator includes all partially folded states in which residue j is folded, and the summation in the denominator includes all partially folded states in which residue j is not folded. In general, the residues with the highest stability constants belong to the folded regions of the most probable partially folded conformations. For those residues, ΣP_{fj}>>ΣP_{nfj} and also Pᵤ>>ΣP_{nf,j}, the above equation essentially reduces to (P_{N}+ΣP_{fj})/Pᵤ. For these residues, the stability constants are larger in magnitude than the global unfolding constant (P_{N}/P_{U}); i.e., they exhibit “super-protection” as observed experimentally (Swint-Kruse & Robertson, 1996).

To illustrate the effect of a mutation to any arbitrary residue, k, on the stability of residue j, the summations can be subdivided further so as to include separately those states in which residues j and k are either folded and unfolded together or individually:

$${κ}_{f , j} = \frac{{P}_{N} + \sum {P}_{f , j \vert f , k} + \sum {P}_{f , j \vert n f , k}}{{P}_{U} + \sum {P}_{n f , j \vert f , k} + \sum {P}_{n f , j \vert n f , k}} .$$

Upon mutation of residue k (i.e., changing the free energy of all states in which residue k is folded) by an amount, Δg_{f,k}(=−RT·lnΦ_{f,k}), the equation becomes

$${κ}_{f , j}^{Mutk} = \frac{{Φ}_{f , k} \cdot {P}_{N} + {Φ}_{f , k} \cdot \sum {P}_{f , j \vert f , k} + \sum {P}_{f , j \vert n f , k}}{{P}_{U} + {Φ}_{f , k} \cdot \sum {P}_{n f , j \vert f , k} + \sum {P}_{n f , j \vert n f , k}} .$$

For the situations considered here and most situations found in the laboratory, Δ_{gf,k} is usually <2 kcal/mol, which is equivalent to a Φ_{f,k} factor as high as 30. Under those conditions, the stability constants of the most stable residues will be affected by mutations occurring anywhere in the protein because the effect of Φ_{f,k} will be seen primarily in the numerator.

The least stable residues, on the other hand, have a relatively high probability of being unfolded in states other than the unfolded state. For these residues, ΣP_{nf,j}>>P_{U}, and Φ_{f,k} in the numerator and denominator will cancel, leaving the stability constant unaffected. In general, the least stable residues are the least affected by mutations anywhere in the protein. From a mathematical point of view the situation can be generalized as follows: The magnitude of the cooperative response of any given residue in the protein is determined by the ratio (P_{U}+ΣP_{nf,j\nf,k})/ΣP_{nf,j\f,k}. In other words, under native conditions, cooperativity will be reflected in the ratio of the probability of global unfolding to the probability of local unfolding. The higher this ratio, the larger the cooperative effect. For this reason, cooperative effects increase under increasing denaturing conditions and are expected to be maximal at the transition midpoint.

C. Donor Cooperativity.

Unlike the cooperative response discussed in the previous section, it can be said that no single residue is able to affect all other residues in the protein. Mathematically, this observation is due to the fact that residues for which ΣP_{nf,j}>>P_{U} will only be affected by mutations when a large penalty exists for unfolding one residue without unfolding the other (i.e., ΣP_{nf,j\nf,k})>>ΣP_{nf,j\f,k}). Thus, only in the case in which the stability of residue k is completely coupled to every other residue in the protein will that residue be able to affect all other residues. As the complete coupling of one residue to all other residues implies a complete coupling between all residues, such a situation exists only for the case of a true two-state transition.

IV. MPMOD

MPMOD utilizes combinations of random searches of conformational space in the allowed regions of Ramachandran plot. The use of these random searches of conformational space provides a simple and useful tool to study the behavior of mini cyclic peptides. This is done using the simple hard sphere model to generate the stereochemically acceptable conformers and flexible disulfide bond modeling, . The “rate” for SS bond loop closure as defined by N_{c}/Nₒ (where N_{c} is the number of conformers that can potentially form disulfide bond and Nₒ is the number of conformers that can not form a disulfide bond but have passed van der Waals check) becomes saturated when the ensemble has more than 1000 conformer. For the CXC and CXXC series of peptides, the modeled probability of loop closure behaves the same way as the experimentally determined equilibrium constant K_{c} for all the four types of the mini peptides. Both compare well after a common scale factor is applied. van der Waals interactions play a dominant role in loop closure for the small peptides CXC and CXXC.

The program (MPMOD) is an efficient method to generate disulfide bonded conformers. It takes about 10˜20 CPU minutes to obtain 4000 disulfide bonded conformers CXXC using a Linux system on a Pentium III 450. Because the conformer CXC has higher probability of collision, it takes about 3 times more CPU time than to generate the CXXC. However, the consumed CPU time strongly depended on the criteria used to generate the conformer.

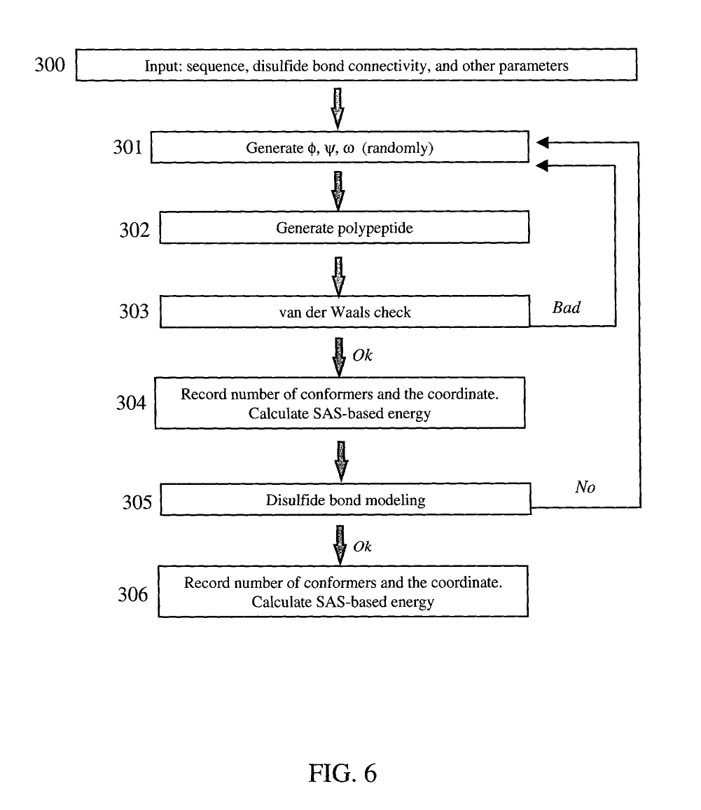

The flow chart illustrated in FIG. 6 illustrates the general program for MPMOD. The input parameters (step 301), such as the peptide sequence and disulfide bond connectivity, are loaded, then the conformational angles (φ, ψ, ω) are generated in the four maps (step 302). The atoms of main chain and side chain are generated base on the angles. The van der Waals checks are performed separately for the backbone atoms and side chain atoms (step 303). If there is a van der Waals violation, the conformer will be rejected. It will go back to get another set of conformational angles until the peptide is finished without any atom collisions. Then the coordinates of peptide are recorded and the solvent accessible surface (SAS) based energy is calculated (step 304). The disulfide bond is modeled to see if there is a disulfide bond is possible for the two residue pairs (step 305). If a disulfide bond is possible, the SAS energy for this conformer is calculated. If a disulfide bond is not possible, another set of conformational angles is tried and the procedure is repeated until a conformer with a disulfide bond is obtained. Finally, the SAS energy is calculated for this conformer again (step 306).

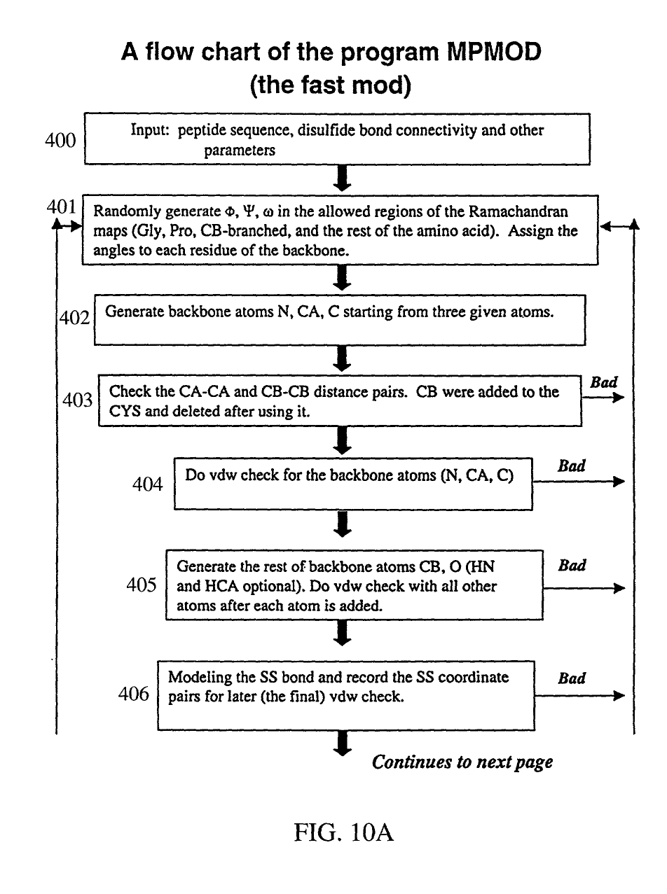

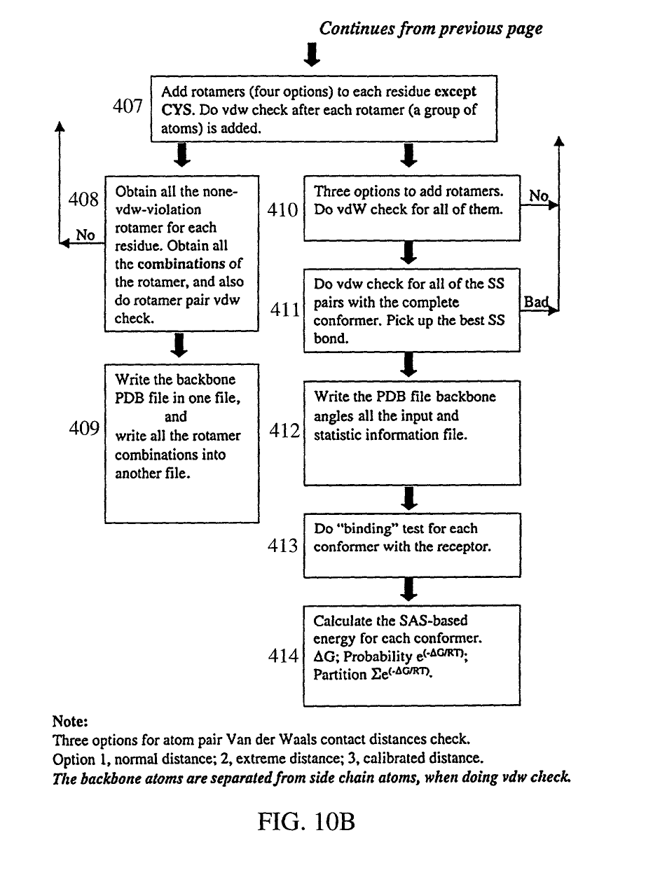

The MPMOD program is designed to generate disulfide bonded conformers or generate disulfide bonded conformers and linear conformers. If the program is run only to generate disulfide bonded conformers, then it is considered “the fast mod”, which is illustrated in FIG. 10A and FIG. 10B. In step 400, sequence, disulfide bond connectivity, and other parameters are inputted. The starting data is inputted manually or it may be retrieved from a database that is well known and used by those of skill in the art. From the input data, dihedral angles (φ, ψ, ω) are randomly generated in step 401 and angles are assigned to each residue of the backbone. The generated dihedral angles are used to generate a backbone atoms, starting from three given atoms in step 402. The distance pairs are checked in step 403. It is important to determine the distance between the two Cα atoms and the distance between the two Cβ atoms. If the distance is not acceptable, then the dihedral angles are regenerated. The distance between the cysteines (C) plays a role in the rate of loop closure. If the distance is acceptable, then a van der Waals check is performed in step 404. If the van der Waals check is acceptable, then the rest of the backbone is generated in step 405. If the van der Waals check is not acceptable, then dihedral angles are regenerated. While generating the backbone, if the van der Waals check remains acceptable, then modeling of the disulfide bonds is performed in step 406. If the van der Waals check does not remain acceptable, then dihedral angles are regenerated. Next, rotamers or side chains are added to the backbone in step 407. Rotamers are added to each residue except for the cysteines. From step 407, one can collate all the none-van der Waals violations in step 408 and regenerate dihedral angles and in step 409 the backbone and all rotamer combinations are written to a file. If the van der Waals check is acceptable for each rotamer in step 410, then disulfide bonded pairs are checked to ensure that the sulfer atom (S) is in good geometry with all the other atoms in step 411. If all the checks are acceptable, then the backbone angles and other information are written to a file in step 412. Next, a binding test is performed in step 413 for each conformer with the receptor to determine which conformer has a higher binding affinity. Finally, the SAS-based energy is calculated in step 414.

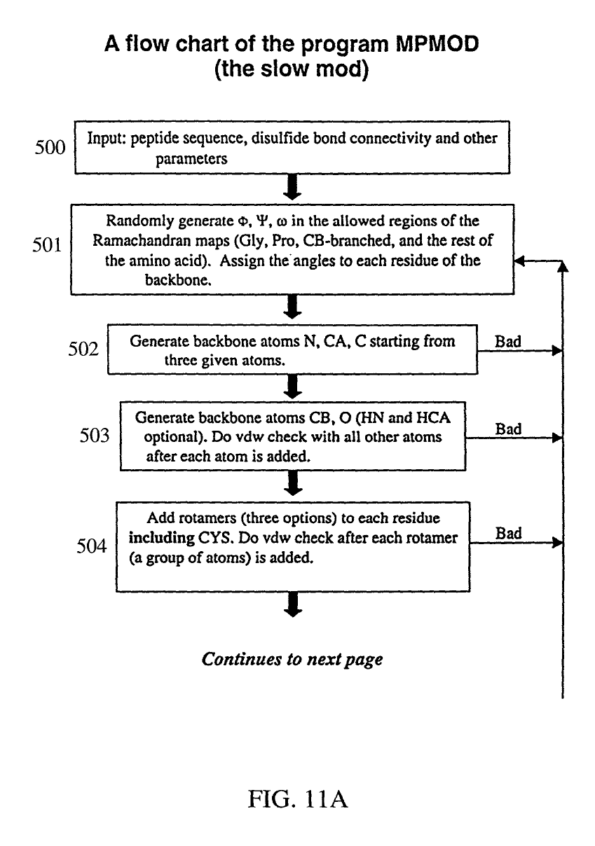

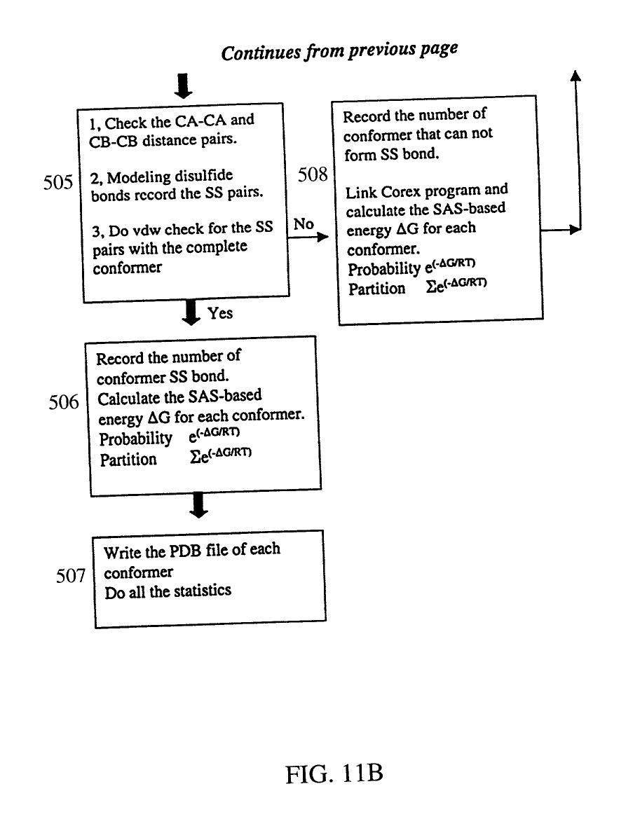

As mentioned the MPMOD program also can generate disulfide bonded conformers and linear conformers. This type of program is considered “the slow mod”, which is illustrated in FIG. 11A and FIG. 11B. In step 500, sequence, disulfide bond connectivity, and other parameters are inputted. The starting data is inputted manually or it may be retrieved from a database that is well known and used by those of skill in the art. From the input data, dihedral angles (φ, ψ, ω) are randomly generated in step 501 and angles are assigned to each residue of the backbone. The generated dihedral angles are used to generate a backbone atoms, starting from three given atoms in step 502. Next, the rest of the backbone is generated in step 503. If the van der Waals check is acceptable, rotamers or side chains are added to the backbone in step 504. Rotamers are added to each residue. After the rotamers are added, the distance pairs are checked, modeling of the disulfide bonds and van der Waals check for the SS paires with the complete conformer in step 505. If any step in 505 is unacceptable, the number of the conformer that can not form a SS bond is recorded and the program is linked to COREX program to calculate the SAS-based energy ΔG for each conformer in step 508. If all steps in step 505 are acceptable, then the number of the conformer SS bond is recorded, the SAS-based energy ΔG for each conformer is calculated in step 506. After the calculations, each conformer is written to a file in step 507.

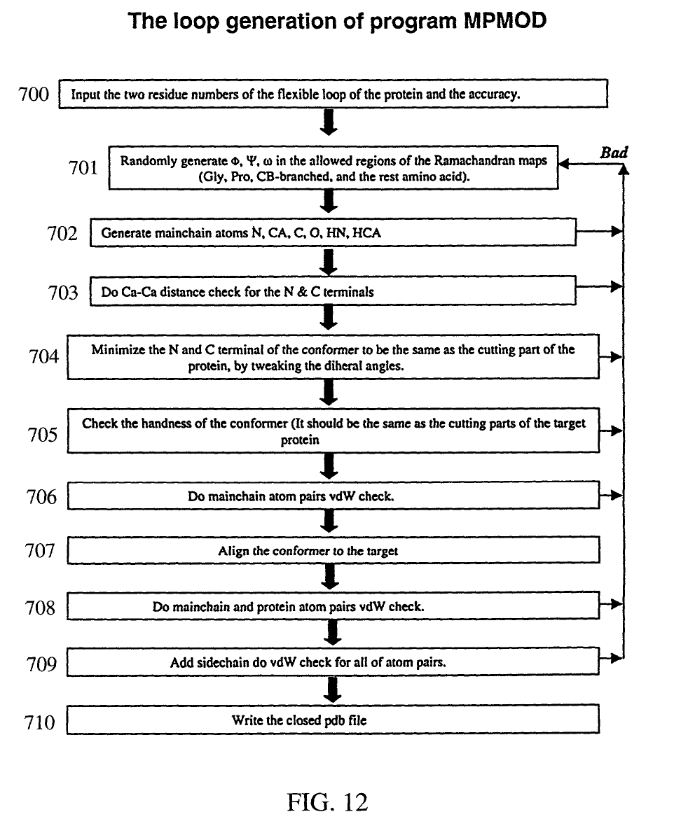

Yet further, the MPMOD program is capable of performing loop generation as shown in FIG. 12. In step 700, two residue numbers of the flexible loop of the protein and the accuracy are inputted. The starting data is inputted manually or it may be retrieved from a database that is well known and used by those of skill in the art. From the input data, dihedral angles (φ, ψ, ω) are randomly generated in step 701 and angles are assigned to each residue of the backbone. The generated dihedral angles are used to generate a backbone atoms or mainchain atoms. The distance pairs are checked in step 703. It is important to determine the distance between the two Cα atoms and N and C terminals of the conformer. In step 704, the distance between N and C terminal of the conformer is minimized by altering or modifying the dihedral angles. Step 705 requires that the handness of the conformer be same as the cutting parts of the target protein. van der Waals check is performed in step 706 of the mainchain atom pairs. If the van der Waals check is acceptable, then the conformers are aligned to the target protein in step 707. van der Waals check is performed in step 708 of the mainchain and target protein atom pairs. If it is acceptable, then rotamers or side chains are added to the mainchain in step 709. If the van der Waals check is acceptable for each rotamer in step 709, then information is written to a file in step 710.

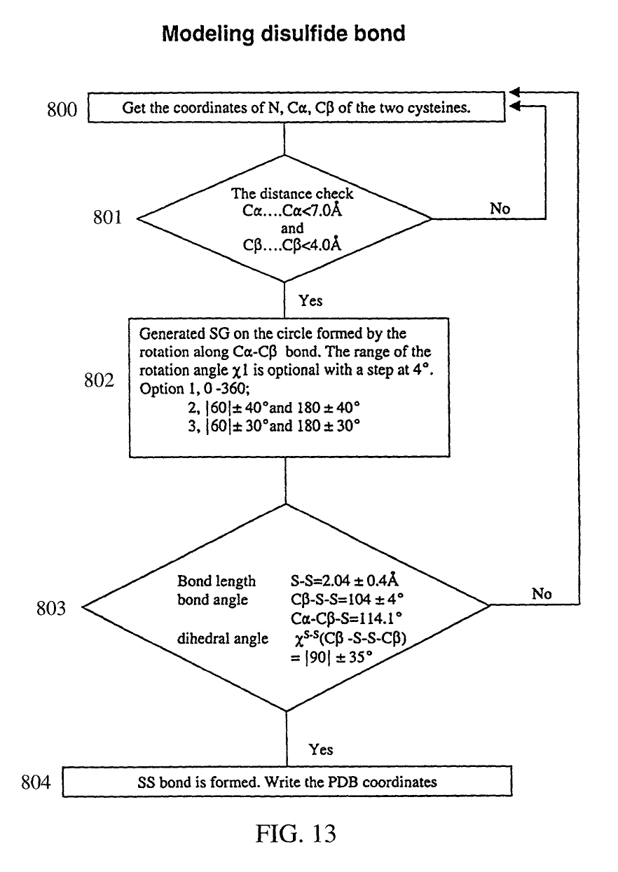

For the disulfide bond modeling module of the MPMOD program is illustrated in FIG. 13. In step 800, coordinates of N, Cα and Cβ of the two cysteines are obtained. Next, in step 801, a distance check is performed for Ca to Ca and Cb to Cb. If the distance is not acceptable, then other coordinates must be obtained in step 800. If the distance is acceptable, then the SG is generated on the circle formed by the rotation along Ca—Cb bond in step 802. Next, bond length, bond angle, and dihedral angles are determined in step 803. If the measurements in step 803 are acceptable, the disulfide bond is formed and the coordinates are written to a file in step 804.

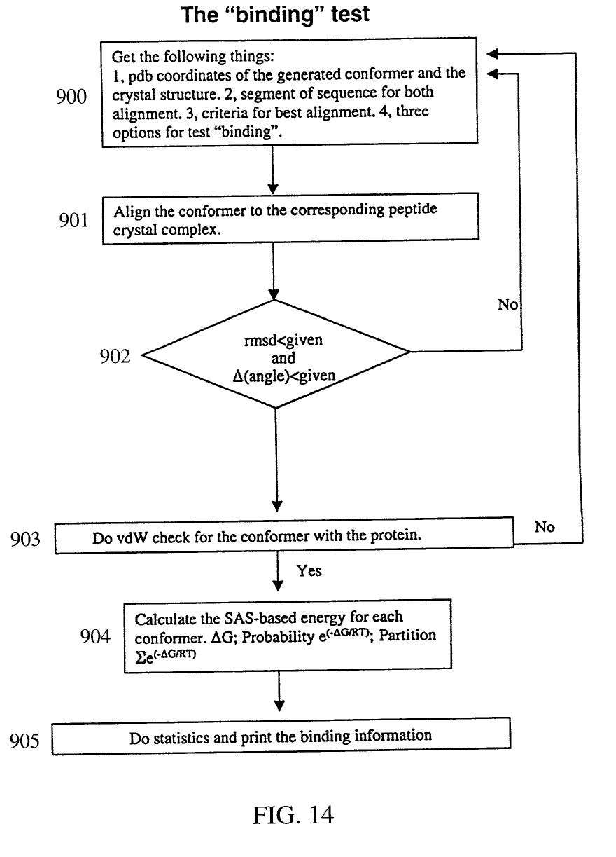

The binding test module of the MPMOD program is illustrated in FIG. 14. Step 900, requires pdb coordinates of the generated conformers and the crystal structure, segment of sequence for both alignments, criteria for best alignments, and three options for test “binding”. Once all the information is gathered, the conformer is aligned to the corresponding peptide crystal structure in step 901. Next, the root mean square deviation between each modeled conformer and the target peptide is determined and the average of conformational angle difference between each residue of the two conformers is determined in Step 902. If the values are acceptable, then van der Waals check of each conformer with the protein is performed in step 903. If the van der Waals check is acceptable, then the SAS-base energy for each conformer is calculated in step 904 and the statistics are preformed in step 905.

V. BEST/MPMOD to Optimize Pharmaceutical Properties

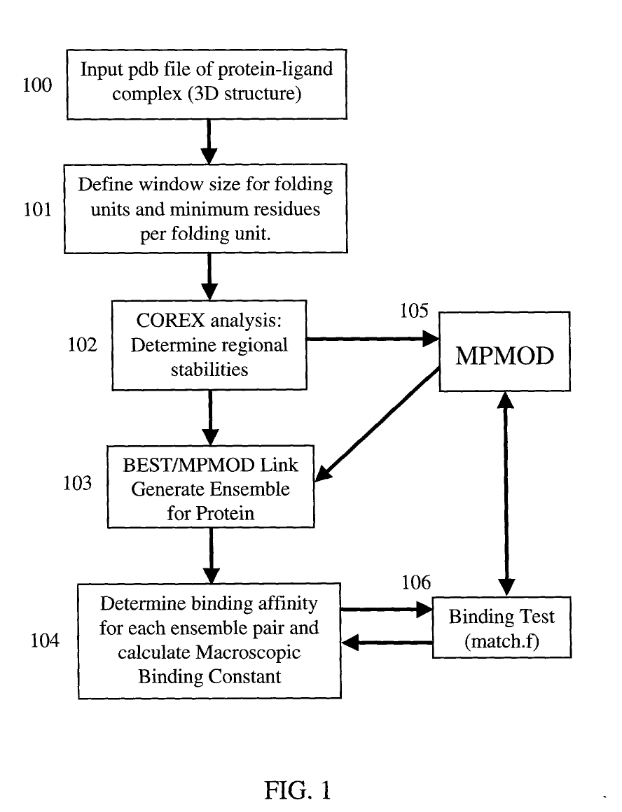

As used herein, “BEST” refers to a technology that models states in a combined fashion. For a given state, regions that are folded are modeled according to the high-resolution structure, while regions that are unfolded are modeled as having a conformational entropy (as opposed to being modeled as a large number of microscopic conformations). As the entropy change (ΔS) is related to the difference in the number of conformational states in the particular macroscopic state relative to the degrees of freedom in the reference of fully folded state, a large number of microscopic states can be represented by a single macroscopic state. The implication of this dual modeling procedure is that a macroscopic state that has 10 residues unfolded (with a minimum of 4 conformations per residue) would require modeling over 1 million explicit microscopic conformational states. The end result of the calculation is that with 100,000 macroscopic states in the ensemble, the present invention effectively captures the energetics of over 10⁶⁰ different microscopic states. The mini-protein modling of disulfides (MPMOD) portion of the software explicitly models conformations for small regions of the protein that are shown to be unstable by BEST. Once the unstable regions are identified using BEST, the two flanking residues of the unstable region will be anchored by fixing the residues in the conformations found in the high-resolution structure. Conformations will be generated by projecting the loop as described in MPMOD. The combination of the BEST and MPMOD programs, termed BEST/MPMOD, allows the explicit modeling of conformations for proteins of any size (FIG. 1). Thus, as shown in FIG. 1, step 100 includes inputting a three-dimensional structure of a protein-ligand complex. The structure can be inputted directly or it may be obtained for any database well known and used by those of skill in the art. Next, step 101 involves defining windso size for folding units and minimum residues per folding unit. Step 102 comprises performing COREX analysis to determine regional stablilities. Step 103 is linked to step 105 which is MPMOD which is linked to step 103, the BEST/MPMOD module to generate an ensembe for the protein. Next, step 104 includes determining binding affinity for each ensemble pair and calculating macroscopic binding constant, which is linked to step 106, which is a binding test module. The binding test module is linked to MPMOD, or step 105.

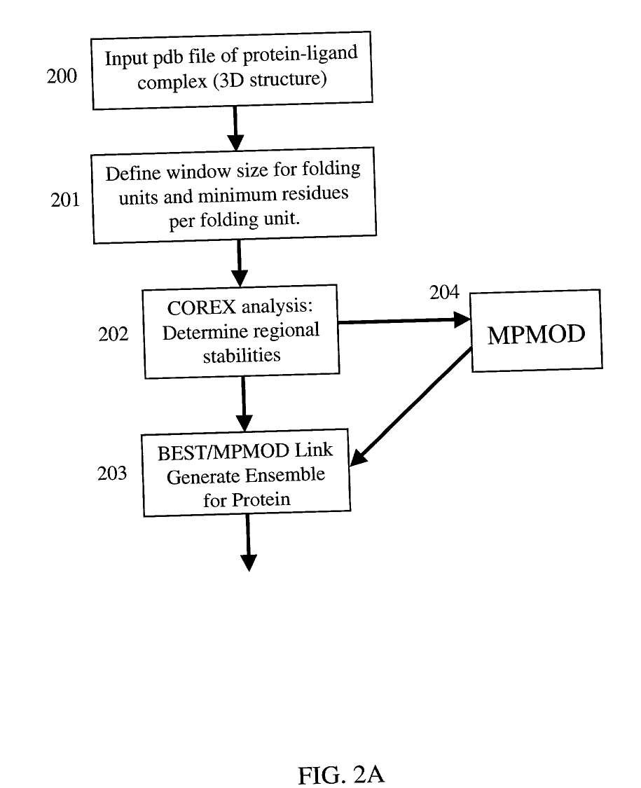

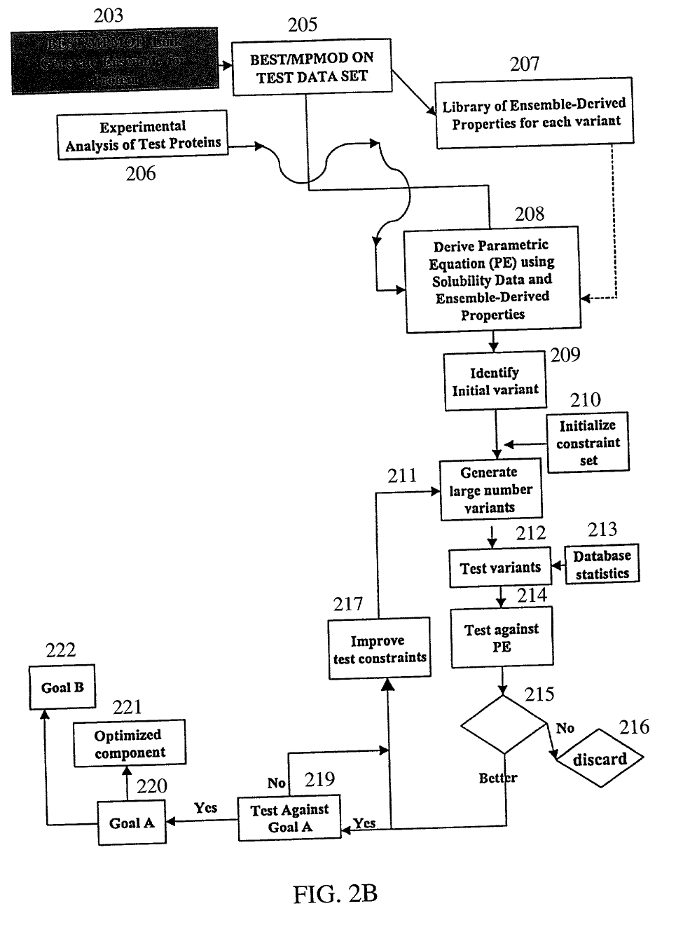

The BEST/MPMOD method of designing a protein pharmaceutical to exhibit optimized pharmaceutical properties as shown in FIGS. 2A and 2B. Step 200 is inputting a high resolution structure of said protein pharmaceutical into a computer-assited modeling program. Step 201 comprises defining a window size for folding units. COREX analysis is performed in step 202 and MPMOD is performed in step 204. The combination of these programs results in the generation of a test data set in step 205. Data are obtained by the following steps: obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; and determining a fraction of conformations of the protein exhibiting optimized pharmaceutical delivery properties. In step 207, the step comprises mutating the amino acid sequence of the protein pharmaceutical to provide a variant and repeating the steps from determining pharmaceutical properties through determining a fraction of conformations a given number of cycles in order to prepare a library of ensemble-derived properties for each variant. Step 208 is deriving a parametric equation using the pharmaceutical delivery properties of each variant and the library of ensemble-derived properties. Next, an initial lead variant is identified in step 209. In step 210, a constraint set is initialized. Based on step 209, a large number of variants is obtained in step 211. Next, in step 212, variants are tested to select a lead mutation set based on a database. Step 214, is testing the lead in the parametric equation. Next, step 215 is to determine if the lead has optimized pharmaceutical delivery properties over the prior lead tested. In step 219, the variant is compared to the goal for each property, Goal A (220) or Goal B (222). Step 221, is to create the variant of the protein pharmaceutical with the structural characteristics found by the above steps to provide optimized pharmaceutical delivery properties. Optionally, the steps from obtaining a large number of variants through determining if the lead is optimized can be repeated to sufficiently optimize the pharmaceutical properties of the protein for its intended pharmaceutical use. Pharmaceutical properties can include but are not limited to, increased binding affinity, decreased aggregation, increased solubility, and decreased immunogenic effects. One of skill in the art realizes that the BEST/MPMOD method of protein design is not limited to protein pharmaceuticals. For example, but not limited to the use of the BEST/MPMOD method to design proteins that may be beneficial as a pesticide or herbicide.

The computer assisted programs of the present invention can be combined with a variety of databsases that would eliminate guess work or decrease the amount of time that is necessary for the programs to run. One such database that may be used is a database that defines thermodynamic propensities of amino acids (See U.S. Provisional Application No. 60/261,733 and U.S. Nonprovisional application No. ______ filed on Jan. 15, 2002, both of which are incorporated in their entirety by reference).

The generating of an ensemble of incrementally different conformations by combinatorial unfolding of a set of predefined folding units in all possible combinations comprises dividing the protein into folding units by placing a block of windows over the entire sequence of the protein and sliding the block of windows one residue at a time, as in step 201.

The determining the probability of each conformational state comprises determining the free energy (Gᵢ) of each of the conformational states in the ensemble; determining the Boltzmann weight [Kᵢ=exp(−Gᵢ/RT)] of each state; and determining the probability of each state using the equation

$${P}_{i} = \frac{{K}_{i}}{\sum {K}_{i}^{′}} .$$

The protection factors are defined by

$${PF}_{j} = \frac{{P}_{fj} - {P}_{fxcj}}{{P}_{nfj} + {P}_{fxcj}}$$

wherein f=folded and nf=not folded and xc=exchange competent.

Determining the energetic connectivities between different structural elements comprises determining the residue-specific connectivities and the functional connectivities.

The residue-specific connectivity is calculated by the equation

$$R S C ( j , k ) = \frac{⟨ ( {S}_{j} - {\overline{S}}_{j} ) \cdot ( {S}_{k} - {\overline{S}}_{k} ) ⟩}{{( ⟨ {( {S}_{j} - {\overline{S}}_{j} )}^{2} ⟩ \cdot ⟨ {( {S}_{k} - {\overline{S}}_{k} )}^{2} ⟩ )}^{1 / 2}} ,$$

wherein Sⱼ and Sₖ denote the folding state of residue j and k (folded S=1, unfolded S=−1) and \overline{Sⱼ} and \overline{Sₖ} denote the average folding state of residues j and k over the ensemble.

The all-atom computational approach is Mini-Protein Modeling (MPMOD) and comprises: searching of conformational space in the allowed regions of the Ramachandran plots; eliminating grossly improbable conformers by a hard sphere approximation; searching flexible disulfide bond models; and calculating a solvent accessible surface (SAS) based energy. The method may further comprise calculating the probability of the ensemble forming a disulfide.

The all-atom computational approach may be Mini-Protein Modeling (MPMOD) and comprises: searching of conformational space in the allowed regions of the Ramachandran plots; minimizing the N and C termini of the conformer to be the same as the high resolution structure; checking the handedness of the conformer; aligning the conformer to the high resolution structure; and performing a van der Waals calculation. The method may further comprise calculating the probability of generating a loop.

Any type of logic may be used to determine which mutations to make to create the data set. Types of logic include but are not limited to any Monte Carlo weighted selection procedure and neural networks.

The present invention may be used for analysis or predictive purposes, depending upon the number of program cycles that are performed. The number of cycles of improvement of pharmaceutical properties that the program performs can be one or more. The number of cycles required to obtain optimized pharmaceutical properties is protein dependent. The steps from obtaining a large number of variants through determining if the lead is optimized may be repeated until the pharmaceutical properties do not improve for a given number of repeats. This given number of repeats may be, but is not limited to, 50 repeats. Any or all of the pharmaceutical properties may be considered in determining if the overall properties have been optimized. For example, for a certain protein pharmaceutical, it may be more important to have a high binding affinity than to have high solubility. Therefore, a lower solubility variant may be accepted in order to have a variant with high binding affinity.

In this invention, any type of commercially available data analysis may be used to get the relationship between the calculated and experimental data. An overdetermined data set is required to perform the invention. An arbitrarily set, protein dependent, number of mutations are examined for any pharmaceutical properties of interest to provide a data set and a “jackknife” analysis if performed on the data set. In the “jackknife analysis”, a part of the data set is removed and the data reanalyzed. If the analysis is not statistically different, then the data set is overdetermined. Any other statistical method of testing whether the data set is overdetermined may be used in this invention. Examples of statistical methods that may be used for analyzing the data are principle component analysis and singular value decomposition. It is possible that the data set for one pharmaceutical property may be overdetermined while the data set for another pharmaceutical property is underdetermined. If a data set is underdetermined, it is necessary to add more mutation variants to the data set

A. Binding Affinity

Binding affinity is the measure of the overall free energy of the interaction between the protein and the ligand. The magnitude of the affinity determines whether a particular interaction is relevant under a given set of conditions. Whether or not any particular affinity of a protein for a ligand is significant depends on the concentration of the ligand present for the protein to encounter. Assays for determining binding affinity include, but are not limited to, surface plasmon resonance, Western blot, ELISA, DNase footprinting, and gel mobility shift assays. The ligand may be protein or non-protein. The ligand may be, but is not limited to, a receptor, a coenzyme, or a non-proteinaceous chemical compound. Binding affinity between a protein and ligand may be measured by the association or dissociation constant of the binding between the protein and the ligand. Entropy of binding between the protein and ligand may be decreased by stabilizing structures similar to that of the protein in a bound state with the ligand. van der Waals calculations can be performed with the protein and the ligand to determine whether binding conformation will be sterically allowed.

B. Aggregation of Proteins

Protein aggregation refers to the interaction of proteins, usually non-specific, to form a complex that may or may not be covalently linked. Aggregation can occur as a competing reaction to folding. Aggregation often causes irreversible precipitation and in vivo can lead to degradation of the complex. Aggregates may form due to exposed hydrophobic areas on partially folded proteins. This may occur with any exposed hydrophobic region, even in a folded protein. Aggregation is a problem in the production of recombinant proteins. This is troublesome in the production of peptides and proteins for pharmaceutical use. Aggregation of proteins or peptides in solution can be determined by measuring light scattering at 360 nanometers as well as by analytical centrifugation. Glutamine/asparagine amino acid rich domains within a protein have been shown to predispose a protein to aggregation.

C. Solubility of Proteins

The solubility of a protein is the amount of the protein that can be dissolved in a given volume of a solvent. The presence of greater than this amount of the protein will cause the protein to aggregate and precipitate. The solubility of a protein in water is determined by its free energy when surrounded by aqueous solvent relative to its free energy when interacting in an amorphous or ordered solid state with any other molecules that might be present, or when immersed in membranes. A factor in the solubility of any substance is the amount of energy required to displace the buffer to accommodate the substance. Ionic strength, pH and temperature of the buffer affect the solubility of a protein. Increasing the ionic strength of the buffer at low values tends to increase solubility of the protein, while increasing ionic strength at high values tends to decrease solubility. In a low ionic strength buffer, the protein is surrounded by an excess of ions of charge opposite to the net charge of the protein. This decreases the electrostatic free energy of the protein and increases solubility. In an aqueous solvent, charged and polar groups on the surface of the protein interact favorably with water. Organic solvents tend to decrease the solubility of proteins. A protein is least soluble at its isoelectric point. At a pH above the isoelectric point, the protein is deprotonated and soluble. At a pH below the isoelectric point, the protein is protonated and soluble. The greater the net charge on a protein, the more likely they are to stay in solution. This is due to the greater electrostatic repulsions between molecules. High temperature causes proteins to denature, thus aggregating and losing solubility.

4. Immunogenicity of Proteins

The immunogenicity of a protein is based upon it binding to proteins of the major histocompatibility complex (MHC). Factors which decrease the likelihood of that occurrence decrease immunogenicity. MHC molecules present the antigen to antibodies. T cells recognize peptide/MHC complexes in the adaptive immune response to antigens. A protein pharmaceutical that is bound by the MHC will not arrive at its site of effectiveness, nor will future molecules of the protein pharmaceutical. Therefore, it is a key objective to design protein pharmaceuticals with low immunogenicity. A smaller protein is less likely to be recognized by the MHC. Therefore, aggregates of a protein can cause increased immunogenicity. In addition, aggregates can trigger degradation which will allow recognition of parts of the protein which are normally inaccessible within the folded protein. Therefore an increase in the stability of a protein will aid in decreasing immunogenicity.

VI. Mutagenesis

Where employed, mutagenesis will be accomplished by a variety of standard, mutagenic procedures. Mutation is the process whereby changes occur in the quantity or structure of an organism. Changes may be the consequence of point mutations that involve the removal, addition or substitution of a single nucleotide base within a DNA sequence, or they may be the consequence of changes involving the insertion or deletion of large numbers of nucleotides.

Structure-guided site-specific mutagenesis represents a powerful tool for the dissection and engineering of protein interactions (Wells, 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Other methods of site-directed mutagenesis are disclosed in U.S. Pat. Nos. 5,220,007; 5,284,760; 5,354,670; 5,366,878; 5,389,514; 5,635,377; and 5,789,166.

VII. Modified Polypeptides

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

In making modifications, the polarity of amino acid residues may be considered. Polar amino acid residues may include: lysine, arginine, histidine, aspartic acid, glutamic acid, asparagine, glutamine, serine, threonine, and tyrosine. Nonpolar amino acid residues may include: alanine, glycine, valine, leucine, isoleucine, proline, phenylalanine, methionine, tryptophan, and cysteine (Alberts et al., 1994).

A. Altered Amino Acids

The present invention encompasses the synthesis of peptides and polypeptides, via transcription and translation of appropriate polynucleotides. These peptides and polypeptides will include the twenty “natural” amino acids, and post-translational modifications thereof. However, in vitro peptide synthesis permits the use of modified and/or unusual amino acids.

B. Mimetics

The present inventors contemplate that structurally similar compounds may be formulated to mimic the key portions of peptide or polypeptides. Such compounds may be termed peptidomimetics.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids. Weisshoff et al. (1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

VIII. Rational Drug Design

The goal of rational drug design is to produce structural analogs of biologically active compounds. By creating such analogs, it is possible to fashion drugs which are more active or stable than the natural molecules, which have different susceptibility to alteration or which may affect the function of various other molecules. In one approach, one would generate a three-dimensional structure for the protein or a fragment thereof. This could be accomplished by X-ray crystallography, computer modeling or by a combination of both approaches. An alternative approach involves the random replacement of functional groups throughout the protein, and the resulting affect on function determined.

It also is possible to isolate a protein specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using an antibody as the antigen.

Thus, one may design drugs which have enhanced and improved biological activity for a given condition relative to a starting structure of the protein. In addition, knowledge of the chemical characteristics of these compounds permits computer employed predictions of structure-function relationships.

IX. Screening Assays

A quick, inexpensive and easy assay to run is an in vitro assay. Various cell lines can be utilized for such screening assays, including cells specifically engineered for this purpose. Depending on the assay, culture may be required. Alternatively, molecular analysis may be performed, for example, looking at protein expression, mRNA expression (including differential display of whole cell or polyA RNA) and others.

In vivo assays involve the use of various animal models, including transgenic animals. Due to their size, ease of handling, and information on their physiology and genetic make-up, mice are a preferred embodiment, especially for transgenies. However, other animals are suitable as well, including insects, nematodes, rats, rabbits, hamsters, guinea pigs, gerbils, woodchucks, cats, dogs, sheep, goats, pigs, cows, horses and monkeys (including chimps, gibbons and baboons). Assays of protein pharmaceuticals may be conducted using an animal model derived from any of these species or others.

In such assays, one or more candidate substances are administered to an animal, and the activity of the candidate substance(s) as compared to a similar animal not treated with the candidate substance(s) is measured.

Treatment of these animals with candidate substances will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

X. Pharmaceutical Preparations

Pharmaceutical compositions of the present invention comprise an effective amount of one or more proteins or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one protein or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In certain embodiments, the present invention concerns a novel composition comprising one or more lipids associated with at least one protein pharmaceutical. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives such as fatty acids, alcohols, amines, amino alcohols, and aldehydes. Of course, compounds other than those specifically described herein that are understood by one of skill in the art as lipids are also encompassed by the compositions and methods of the present invention.

### XI. EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### The Native State of DHFR is an Ensemble

The energetic connectivities in DHFR was determined through COREX analysis on the crystal structure of the DHFR•folate•NADP⁺ ternary complex [Protein Data Bank accession no. 7dfr (Bystroff et al., 1990)] with the folate and NADP⁺ molecules removed. To achieve a higher-resolution analysis than previously described, the COREX algorithm was modified to use a Monte Carlo sampling strategy (Metropolis et al., 1953). In separate analyses, it was demonstrated that the Monte Carlo sampling provides equivalent results to those obtained with the original COREX sampling.

High stability constants signify residues that are folded in the majority of highly probable states under native conditions, whereas lower stability constants signify residues that are unfolded in many of those states. In general, residues with higher stability constants lie in β strands 1, 6, and 8 and α helices 1, 2, and 4, whereas lower stability constants are found in several of the loop regions that separate the elements of regular secondary structure.

### Example 2

### An Ensemble View of Cooperativity

Cooperativity in proteins is the result of energetic coupling between different regions. Within the context of an ensemble-based description of the equilibrium, cooperativity is manifested in the relative probabilities of the different states in the ensemble. For regions that are highly coupled energetically, the probability of states in which both regions are folded or unfolded is greater than the probability of states in which only one is folded. Since the COREX algorithm provides reasonable estimates for the probabilities of the different states in the ensemble, it captures the network of cooperative interactions in the protein. This fact implies that the cooperativity between different structural and functional elements of a protein can be ascertained through an analysis of those regions of the protein that are folded in the states with the highest probabilities. If two residues, j and k, are both folded or unfolded in the majority of highly probable states, the residue stability constants will be identical for both residues. As such, a perturbation that affects residue j by a specific amount will necessarily affect residue k by that same amount. This reasoning can be extended to investigate the energetic coupling between groups of residues as well. Here the inventors define the coupling between two residues or groups of residues as the connectivity, and the inventors identify two distinct types of connectivity. The first describes how changes at individual residues are propagated (i.e., point mutations), whereas the other describes how changes to groups of residues are propagated (i.e., site-specific binding).

### Example 3

### Residue-Specific Connectivities

The residue-specific connectivity (RSC) is the coupling between two residues. In the context of the Monte Carlo sampling method, RSC is defined by the correlation function:

$$R S C ( j , k ) = \frac{⟨ ( {S}_{j} - {\overline{S}}_{j} ) \cdot ( {S}_{k} - {\overline{S}}_{k} ) ⟩}{{( ⟨ {( {S}_{j} - {\overline{S}}_{j} )}^{2} ⟩ \cdot ⟨ {( {S}_{k} - {\overline{S}}_{k} )}^{2} ⟩ )}^{1 / 2}} ,$$

where Sⱼ and Sₖ denote the folding state of residue j and k (folded S=1, unfolded S=−1) and \overline{Sⱼ} and \overline{Sₖ} denote the average folding state of residues j and k over the ensemble. A positive value of the RSC indicates that a stabilization of residue j or k results in a stabilization of residue k or j (i.e., they display positive cooperativity), whereas a negative value indicates a stabilization of residue j or k leads to a destabilization of residue k or j (i.e., they display negative cooperativity). A value of zero means there is no correlation, and the residues are not energetically coupled.

Because the above equation provides the mutual susceptibility of each residue to perturbations at every other residue, it can be used to probe the thermodynamic domain structure in proteins.

In addition to providing thermodynamic domain assignments, the RSCs can also be used to explore how mutational effects propagate from their point of origin.

One of the hallmark features of DHFR is the experimental observation that mutations at distal regions of the protein often propagate many angstroms through the structure and preferentially affect the affinity of DHFR for either NADPH or folate (Fierke and Benkovic, 1989, Warren et al., 1991, Ohmae et al., 1996, Cameron and Benkovic, 1997, Ohmae et al., 1998). The present invention can be used to measure if a correlation exists between how each binding site is affected by a mutation and the experimentally observed effect on binding. In general, the effect of a mutation on the stability of a binding site should necessarily scale with the change in binding affinity. As shown in Table 2, mutational effects in DHFR do propagate over many angstroms, and the degree to which a particular binding site is energetically coupled to the mutated residue correlates with the effect of that mutation on the affinity for each ligand. Although this correlation clearly provides no mechanistic details about how binding affinity is affected by the mutation, the correlation in behavior supports the accuracy of the connectivity information provided by the residue specific connectivities and demonstrates the ability of the algorithm to map effects over long distances.

### Example 4

### Functional Connectivities

Experimentally, it has been established that binding in either the NADPH- or folate-binding sites affects the affinity of DHFR for the other ligand (Fierke et al., 1987). The nature of this effect can be cast in terms of the ensemble. As noted, the ensemble of DHFR conformations that exist under native conditions in the absence of both folate or NADPH is characterized by states in which many of the loop regions are unfolded, a subset of which are involved in binding to either ligand. In the presence of either ligand, however, those states whose binding-site residues are folded will bind ligand and will be preferentially stabilized over those states in which all or part of the active site is unfolded (Metropolis et al., 1953). Thus, in addition to the pairwise correlations described by the RSCs, it is necessary to identify those joint correlations between the binding site as a whole and the rest of the protein. The functional connectivity (FC) is defined as the connectivity between the entire binding site for a given ligand, x, and every residue in the protein:

FCₓ(j*,k*)=RSC(j,k),

where j* and k* are defined in one of two ways. For all residues, j and k, not involved in the binding pocket for ligand x, Sⱼ*=Sⱼ and Sₖ*=Sₖ in the residue specific connectivity equation. For residues in the binding site for ligand x, however, Sⱼ* and/or Sₖ* are the average folding states over all of the nₓ residues in the binding site for ligand x:

$${S}_{j}^{*} = \frac{{\sum }_{y = 1}^{{n}_{x}} {S}_{j}}{{n}_{x}}$$

The difference between RSCs and FCs is noteworthy. RSCs are determined by correlating the probabilities of states in which two particular residues are folded, independent of the folded state of other residues. FCs, on the other hand, correlate the probability of a residue being folded with the probability of a group of residues being folded. As such, FCs effectively amplify connectivity information that is often not seen in an analysis of the RSCs. This result highlights the difficulties in experimentally deriving the functional connectivities in proteins from the effects of single site mutants.

TABLE 2

| | Distance, Å* | | Relative effect | Relative effect on κ_{f} of |
|---|---|---|---|---|
| Mutants | Folate | NADPH | on Kₘ† | binding-site residues‡ |
| 22 | 14 | 18 | Folate > NADPH | Folate > NADPH |
| 67 | 23 | 17 | NADPH > folate | NADPH > folate |
| 113 | 12 | 18 | Folate > NADPH | Folate > NADPH |
| 121 | 19 | 15 | NADPH > folate | NADPH > folate |
| 145 | 21 | 28 | No effect | No effect |

*The distance between a mutated residue and ligand-binding sites is the average distance
between the mutated residue and all the residues in folate or NADPH-binding site.
† Measured effects of mutation on Kₘ for folate and NADPH (10-18), e.g.,
folate > NADPH means the effect of mutation on Kₘ,
for folate is larger than the effect on Kₘ for NADPH.
‡ The predicted effects of mutations on ligand-binding sites are obtained by averaging RSC
values between the mutation site and each residue in the folate or NADPH-binding site
$( {effect}_{x} = ( {\sum }_{i = 1}^{{n}_{x}} RSC ( j , k ) ) / {n}_{x} ) ,$
where k is the mutated residue, and the summation is over all
nₓ residues in the binding site for ligand x.

The importance of this result is 3-fold. First, the affected loop is more than 15 Å from the folate-binding site, indicating that the ensemble approach adequately models the propagation of binding energy through DHFR. Second, the residues that surround the affected loop show no connectivity to the folate site, indicating that residues can be energetically coupled in the absence of a visible connectivity pathway. This result undermines the mechanical view of signal propagation wherein binding or mutational effects are propagated to distal parts of the protein through a series of conformational distortions. Third, and equally important, one skilled in the art recognizes that these results contradict the classic view that binding “freezes out” protein conformations, resulting in a decrease in motion (or dynamics) (Froloff et al., 1977). The experimental observation that binding can increase dynamics in some systems (Bolin et al., 1982, Bystroff and Kraut, 1991, Akke et al., 1993, Olejniczak et al., 1997, Yu et al., 1996, Stivers et al., 1996, Zidek et al., 1999) emphasizes the importance of entropic contributions. The success of the approach of the present inventors in capturing this effect implies that the entropic contributions are adequately represented in the ensemble view as implemented by the COREX algorithm.

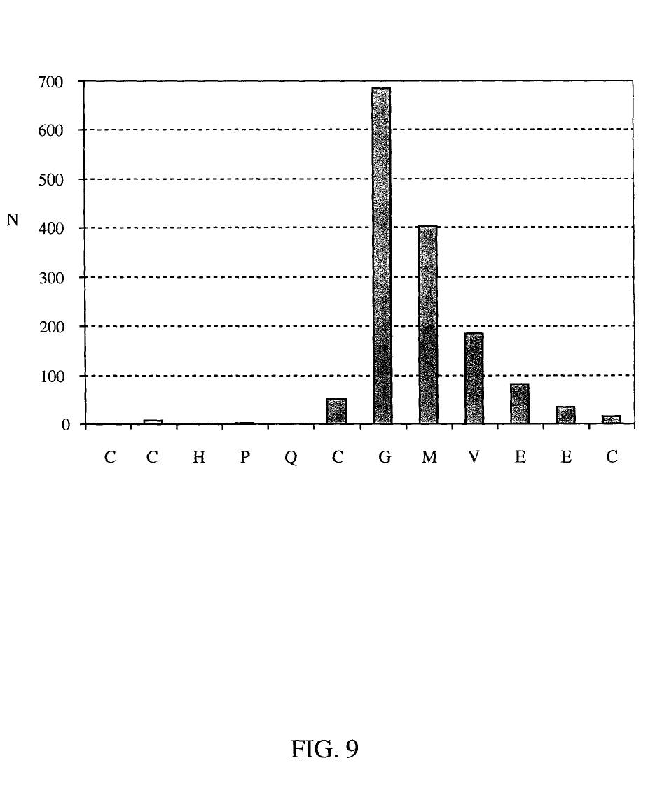

Although the approach presented here contains many simplifying assumptions, which includes the fact that only the structure of the ternary complex (7dfr) was analyzed, the algorithm is nonetheless successful in capturing significant details regarding the energetic connectivities in DHFR. The reason for this success is rooted in the origins of energy propagation. Thus, it is the number and relative probability of the low-energy states that determine the magnitude and extent to which binding and mutational effects are propagated throughout the structure. For DHFR, the differences in Ln κ_{f} between residues 60-90 and the rest of the protein (FIG. 9A) indicate that states with this region unfolded are greater than 7.0 kcal/mol more stable than states with other regions unfolded. Consequently, most mutational effects, which rarely account for more than 2 kcal/mol, are unlikely to significantly affect the energetic hierarchy of states and thus the connectivity pattern. It is this relationship between the distribution of states in the ensemble and the connectivities between the different functional elements that allows proteins to tolerate significant sequence diversity while maintaining complex biological behavior.

### Example 5

### Deriving an Ensemble View of Molecular Recognition Using BEST/MPMOD

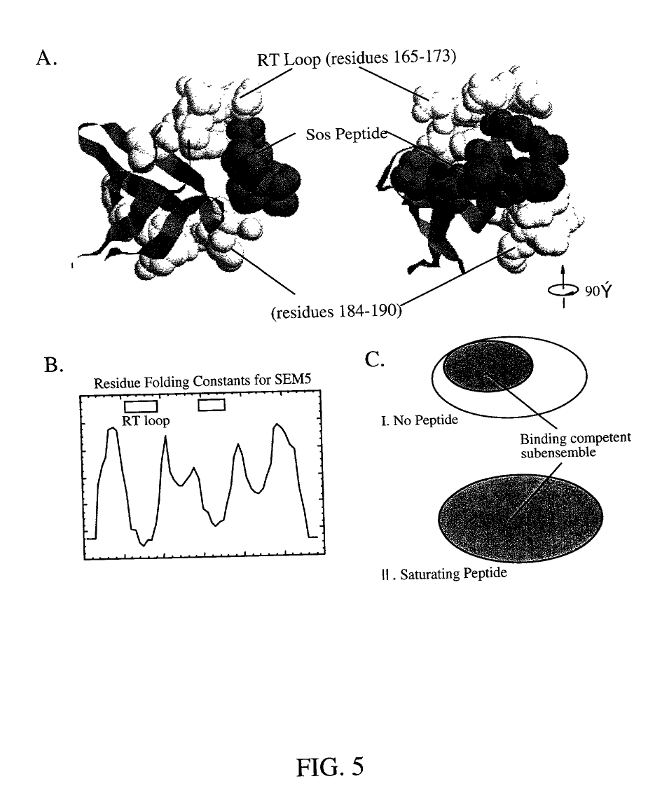

The first step in the development of a quantitative model for binding is recasting molecular recognition in terms of ensembles. FIG. 5A shows the structure of a model system, the Src-homology-3 (SH3) domain of the C. elegans protein SEM5 in complex with the Sos peptide. FIG. 5B shows the pattern of folding constants for SEM5, as calculated by the COREX algorithm. As expected, there are regional differences in the pattern of folding constants. Among the lowest folding constants are those found for residues 165-173 (known as the RT loop) and residues 184-190. As evident from FIG. 5A, a number of residues located in these loop regions make direct contact with the Sos peptide in the SEM5/Sos complex.

An implication of the low stability in the RT loop is that in the absence of peptide, the SEM5 ensemble contains a significant population of molecules which have the loop disordered. As the binding energy for this sub-ensemble will involve the energy necessary for ordering the loop, these states will have a reduced or negligible affinity for peptide. This point is illustrated in FIG. 5C, where the fraction of binding-competent species is represented as a subset of the total ensemble in the absence of peptide. Binding of Sos to SEM5, therefore, requires redistributing the SEM5 ensemble to bias binding-competent species.

Within the context of the ensemble-based view depicted in FIG. 5C, the observed affinity constant, Kobs, of a protein for ligand is a composite of the binding constants of all conformational states in both the protein and the ligand ensembles, and can be represented in matrix form as:

$$( \begin{matrix}{P}_{1} {L}_{1} \\ {P}_{2} {L}_{1} \\ {P}_{m} {L}_{1}\end{matrix} \begin{matrix}{P}_{1} {L}_{2} \\ {P}_{2} {L}_{2} \\ {P}_{m} {L}_{2}\end{matrix} \begin{matrix}{P}_{1} {L}_{n} \\ {P}_{2} {L}_{n} \\ {P}_{m} {L}_{n}\end{matrix} ) + ( \begin{matrix}{P}_{1} + {L}_{1} \\ {P}_{2} + {L}_{1} \\ {P}_{m} + {L}_{1}\end{matrix} \begin{matrix}{P}_{1} + {L}_{2} \\ {P}_{2} + {L}_{2} \\ {P}_{m} + {L}_{2}\end{matrix} \begin{matrix}{P}_{1} + {L}_{n} \\ {P}_{2} + {L}_{n} \\ {P}_{m} + {L}_{n}\end{matrix} ) = Ψ \cdot ( \begin{matrix}{K}_{1 , 1} & {K}_{1 , 2} & {K}_{1 , n} \\ {K}_{2 , 1} & {K}_{2 , 2} & {K}_{2 , n} \\ {K}_{m , 1} & {K}_{m , 2} & {K}_{m , n}\end{matrix} )$$

where K_{i,j} is the microscopic binding constant between the ith protein and jth ligand conformation. K_{i,j} is determined from the energy difference between PᵢLⱼ and the isolated Pᵢ and Lⱼ conformations (denoted as operator Ψ; an additional cratic entropy term −ΔScratic=−R*ln55 is needed to account for the decrease in the degrees of freedom). As the above equation implies, the observed binding affinity of SEM5 to Sos requires knowledge of the structural and thermodynamic attributes of the conformational ensembles of both the Sos peptide and the SEM5 protein. The BEST/MPMOD program addresses these issues.

/******** BEST Calculation at any Temperature ********/
Open_File();
/*opens pdb file*/
read_pdb_file (&atom_list, infile);
/* reads pdb file*/
num_atoms = load_atoms(&atom_list, infile);
/* counts and assigns atoms*/
Enter_Parameters();
/*Enter Entropy scaling,, window size, minimum window size, temperature */
ASA_parameters();
/* Assigns accessible surface area parameters */
asa_calculate(atom_list, num_atoms, slice_width, solvent_radius);
/* calculate ASA for native state */
for(i=0;i<num_atoms;i++) Atom_Area_Native[i]=atom_list[i].area;
fclose(infile);
/*assign atom areas for the native state based on ASA calculation*/
Gen_Partition();
/* define partitioning of protein: combinatorial unfolding of all folding units*/
Initialize_Thermo();
/* Initialize partition function and populations*/
for (partition=1;partition<=Npartition;partition++)
/* for all partitionings */

{
for (j=1;j<Final_State;j++)
/* for each intermediate state in partitioning */

{
int2bin(j);
/* creates binary number from integer values */
num_atoms = load_atoms_range(&atom_list, infile);
/* assign residues that will have surface area calculation for each state */
if (!asa_assign_radii(atom_list, num_atoms, NULL,0))

{
fprintf(stdout, “Radii assignment failure.\n”);
exit(1);
}

Fraction_Folded=(float)(num_residues)/(float)(Total_Res);
ASA_parameters();
/* Assigns accessible surface area parameters */
asa_calculate(atom_list, num_atoms, slice_width, solvent_radius);
/* Calculate ASA for that state*/
for(i=0;i<num_atoms;i++) Atom_Area[i]=atom_list[i].area;
Native_State2();
/* calculate values for folding units in native state */
calc_stat_weight(atom_list, num_atoms);
/* determines the energies and statistical weights of each state*/
State_Probabilities();
/* calculates the statistical weight for each sub-ensemble with different
degrees of folding*/
Residue_Probabilities();
/*determines residues specific stability [i.e., Pj,folded/Pj,notfolded*/
} /* end loop for each intermediate state in partitioning */

} /* end partitions loop */

for(i=1;i<Max_Res;i++)

{
Prob_unfolded25[i]=Prob_unfolded25[i]/Partition_Function25;
/* calculate probability from statistical weight*/
Prob_complement25[i]=Prob_complement25[i]/Partition_Function25;
}

Define_MPMOD_Res();
/*identify residues with probabilities below threshhold value*/
MP_MOD_input(res_list,res_stab);
/*perfom loop generation calculation using MPMOD*/
MP_MOD_slow(Lig_Res);
/* generate ensemble for ligand*/
MP_MOD_Binding_Test();
/*check binding of all ligand ensemble members with all protein ensemble members*/
Save_Probabilities();

### Example 6

### Construction of Peptides Using BEST/MPMOD for Modeling the Peptide-Streptavidin Complex

The backbone dihedral angles (φ,ψ) of the peptide were randomly generated in the four Ramachandran maps, one for glycine, one for proline, one for the CB-branched amino acids (VAL, ILE and THR), and one for all other amino acids. The trans and cis forms of the peptides have dihedral angles of ω=180° and 0° with a small random deviation (usually within ±5°). The backbone of the peptide was generated based on the dihedral angles (φ, ψ, ω) and the standard bond lengths and bond angles. Ponder and Richard's rotamer library (1989) was used to add side chains to the backbone. The simple hard sphere approximation was used to eliminate the grossly improbable conformers. Each atom was thought of as a hard sphere with its appropriate van der Waals radius. The minimum distances (Iijima et al 1987) between two atoms were used for the van der Waals check for each atom pair. These distances are about 0.2 to 0.4 Å shorter than the “normal” distance of Ramachandran et al (1963). If the backbone hydrogen atoms (HN and HCA) are generated, the overlap of the H atom with other atoms is even larger (about 0.5 Å) than normal. Otherwise, it would not be efficient to generate the conformer due to van der Waals violations.

### Example 7

### Modeling Disulfide Bonds Using BEST/MPMOD for the Peptide-Streptavidin Complex

A single disulfide bond can be modeled by the present invention. When two disulfide bonds are modeled in a conformer, attention should be paid to the computational efficiency. The probability of forming two disulfide bonds simultaneously for a polypeptide is the product of the probabilities for each disulfide bond to form. Currently, it takes a long time to generate one peptide with two disulfide bonds. The present invention provides an efficient way to model a two disulfide bond conformer. With two disulfide bonded loops, the short one is modeled first. Conformations of this loop are fixed when the short one forms a disulfide bond. When the first loop is fixed, it may take a long time to find the second loop if the first loop does not have suitable geometry. Therefore, some number of tries must be given to search for the second loop, while the conformation of the first loop is fixed. The number of tries is usually set to be between about 5 and 10. It is possible to obtain several polypeptides with one fixed conformation for the first loop and various conformations for the second loop. All conformers in the ensemble are kept for the “binding” test.

If the polypeptide is cyclized by a lactam covalent bond (i.e., the nitrogen (N) of the first residue makes a covalent bond with the carbon (C) of the last residue) the method for modeling the disulfide bond is no longer valid. The criteria to form such cyclic peptides are 1.35±0.6 Å for the N—C bond length and 120±35° for the bond angles (CA—N—C or CA—C—N). It is less efficient to generate such cyclic peptides than to generate a one disulfide-bonded peptide, since the former is searched only by one position of the atom N or C, whereas the latter is searched from a number of positions of sulfur.

### Example 8

### Aligning the Conformers to the Binding Site of Streptavidin Using BEST/MPMOD

After ensembles of conformers were generated, the “binding” test was performed. The first step is to align the conformer to the template. The template is the peptide in the co-crystal structure complex. The second step is to screen the conformer by using the hard sphere potential model. For the peptide-streptavidin complex, the dominant binding force occurs at the HPQ sequence of the peptide, the modeled conformers were aligned to the corresponding HPQ sequence of the crystal structure of the complex. Any high resolution X-ray crystal structure can be used for the template. Two criteria were used to determine whether or not the alignment is successful. One criterion is the root mean square deviation (rmsd) between each modeled conformer k and the target peptide t.

$$R m s d ( k , t ) = {[ {{\sum }_{j = 1}}^{n} ( {( x ( k , j ) - x ( t , j ) )}^{2} + {( y ( k , j ) - y ( t , j ) )}^{2} + {( z ( k , j ) - z ( t , j ) )}^{2} ) ]}^{\frac{1 / 2}{n}}$$

where n is the number of atoms participating in the alignment (n=9 for the HPQ sequence).

Another criterion is the average of conformational angle difference between each residue of the two conformers.

$$Δ A ( k , t ) = {\sum }_{j = 1}^{m} ( \vert φ ( k , j ) - φ ( t , j ) \vert + \vert ψ ( k , j ) - ψ ( t , j ) \vert ) / ( 2 * m )$$

where m is number of residues for the compared sequence (m=3 for the HPQ sequence).

To determine whether the alignment is acceptable or not, two common reference values rmsd_{ref} and ΔA_{ref}, for rmsd(k, t) and ΔA(k, t) respectively, are given. For the kth conformers in the ensemble, if rmsd(k, t)≦rmsd_{ref} and ΔA(k, t)≦ΔA_{ref} are satisfied, then this aligmnent is acceptable. If any one of the criteria is not satisfied, the alignment is unacceptable and the conformer will be rejected. For the HPQ sequence, the reference values are rmsd_{ref}=0.50 Å (Three atoms Cα, C and N were used for the alignment for each residue.) and ΔA_{ref}=50°.

If the alignment is acceptable, a van der Waals check with streptavidin is performed as the second step to determine whether or not the final docking is successful. If there are any collisions for the atom pair of conformer and the target protein, the docking is not successful and the conformer is rejected. The atom radius for van der Waals check is the same as those mentioned before. If there is no van der Waals violation for any atom pair, the conformer is considered as being successfully docked into the protein. The “binding ratio” can be defined as the ratio N_{b}/Nₜ, where N_{b} is the number of conformers that can be successfully docked into the HPQ binding pocket and Nₜ is the total number of the conformers in the ensemble. The ratio correlated well with the experimentally measured binding affinity of the complex.

### Example 9

### Cluster Analysis of the HPQ Sequence Using BEST/MPMOD

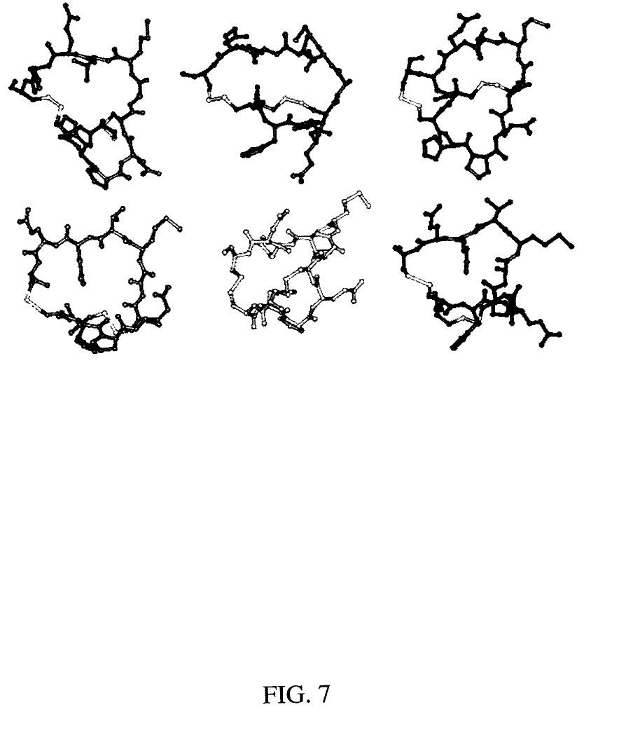

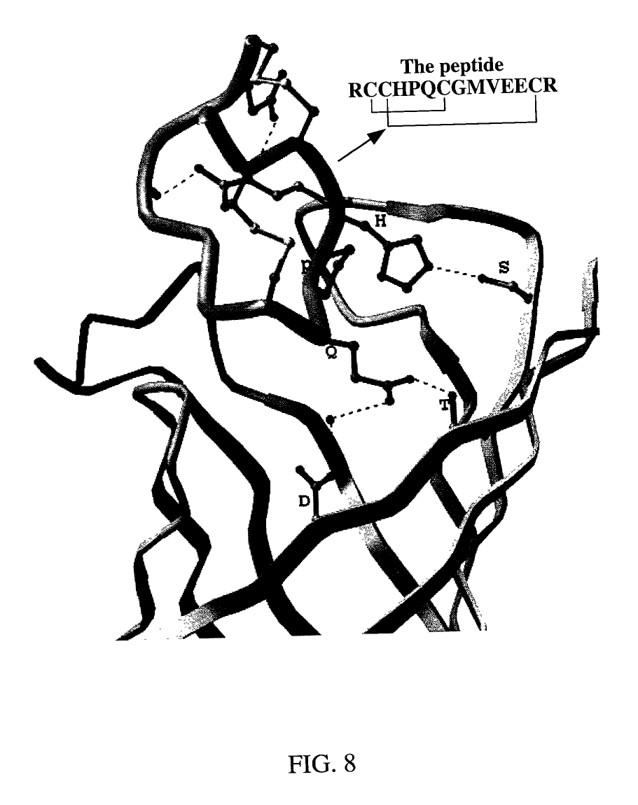

The peptide-streptavidin complex was surveyed. Table 3 lists the peptides and experimentally measured binding affinities with streptavidin. Ensembles of conformers for all these peptides have been generated following the above procedures. FIG. 7 gives an example of the ensemble for the peptide of CCHPQCGMVEEC. The HPQ sequence of the peptide is crucial for binding so it is necessary to know what fraction of the modeled conformers can adopt a type-I β turn in the HPQ sequence. The crystal structure of CCHPQCGMVEEC (FIG. 8), determined at resolution 1.46 Å, was used as the template to calculate that fraction. All the modeled conformers are aligned to the HPQ sequence of the crystal structure CCHPQCGMVEEC, using the reference values rmsd_{ref}=0.50 Å and ΔA_{ref}=50°. For each conformer, if the calculated rmsd(k, t) and ΔA(k, t) are both less than the given reference values, the conformer is said to be HPQ-like, or it is similar to the crystal structure in the HPQ sequence. In other words, the modeled HPQ sequence can adopt a type-I β turn. The percentage of conformers able to satisfy the criteria is listed in Table 3.

TABLE 3
The list of experimentally observed binding constant
and the modeled “bind ratio”(f_{b}).

| | Observed | | Modeled | |
|---|---|---|---|---|
| | K_{D} | K_{A} | HPQ-like | Binder (f_{b}) |
| Peptide | (μM) | (μM⁻¹) | (%) | (%) |
| HDHPQNL¹ | 282 | 0.004 | 4 | 1.3 |
| FSHPQNT¹ | 125 | 0.008 | 6 | 1.4 |
| AHPQFPAEK⁴ | 136 | 0.007 | 6 | 1.50 |
| AHPQFGAEK⁴ | 204 | 0.005 | 6 | 0.85 |
| AECHPQGPPCIEGRK² | 0.23 | 4.35 | 24.9 | 11.3 |
| AECHPQFPCIEGRK² | 0.93 | 1.08 | 41.9 | 28.7 |
| AECHPQFNCIEGRK² | 7 | 0.14 | 22.4 | 11.3 |
| AECHPQFCIEGRK² | 0.47 | 2.13 | 16.1 | 6.6 |
| cyclo-CHPQFC² | 0.27 | 3.70 | 18.7 | 16.9 |
| cyclo-CHPQGPPC² | 0.67 | 1.49 | 23.5 | 12.0 |
| cyclo-(AHPQFPAE)K⁴ | 0.13 | 7.69 | 21.4 | 21 |
| cyclo-(AHPQFGAE)K⁴ | 19 | 0.05 | 12 | 7.1 |
| cyclo-(AQYGHFAE)K⁴ | >5000 | 0.0002 | | |
| RCCHPQCGMVEEC⁵ | 1.3 | 3.3 | 27.8 | 7.5 |
| RCCHPQCGMAEEC⁵ | 2.3 | 0.45 | 25.3 | 7.2 |
| RCCHPQFEPCMGC⁵ | 0.33 | 3.0 | 19.6 | 7.4 |

The HPQ-like conformer for the linear peptides (around 6%) is about 2-7 times smaller than the peptides with a disulfide bond (12%-42%) (Table 3). The reason is that the linear peptides are not restrained in conformational space and can accept various conformations. Whereas, for the peptides with a disulfide bond, the configuration is constrained. The HPQ-like ratio for the linear peptides does not vary much. The ratio for the cyclic peptides varies according to the type and number of amino acids between the two cysteines. The only difference between the conformer AECHPQFNCIEGRK and AECHPQFPCIEGRK is at residue 8. But the ratios for both have a significant difference in which the former has a ratio of 22.4% and the latter has a ratio of 41.9%. Having a proline as residue 8 greatly increased the chance to form a type-I β turn for the HPQ motif.

In the peptides, CCHPQCGMVEEC and CCHPQCGMAEEC the first two cysteines are too close to each other to form a disulfide bond. The combinations of the disulfide bonds that can be formed are the crossed form C1-C6, C2-C12, and the nested form C2-C6, C1-C12. The crossed form has a higher percentage of HPQ-like conformers than the nested form. This was caused by the smaller loop. It is known in the art that the equilibrium constant K_{c} for forming CXXXC is smaller than for forming CXXXXC (Zhang and Snyder, 1989). The first loop in CCHPQC for the crossed form adopts higher ratio of type-I β turn in the HPQ sequence. When Ala is replaced by Val for peptides with two loops, the fraction of HPQ-like conformers increase. The CB branched amino acid further limits the conformation of the HPQ sequence, which enhanced the ratio of HPQ-like conformers.

### Example 10

### The “Binding Ratio” of Peptide-Streptavidin Complex Using BEST/MPMOD

The X-ray co-crystal structure shows that all of the peptides bind to streptavidin at the same site. The HPQ sequence is crucial for the binding of the complex. When the HPQ motif of the modeled conformers is similar to that of the corresponding crystal structure, the modeled conformer has the potential to bind with streptavidin. Each HPQ-like conformer is aligned to the HPQ sequence of the co-crystal structure. If the conformer does not have a van der Waals collision with the target protein, it is defined as a “binder”. The larger the fraction of “binders” in the ensemble, the higher the binding affinity is for the complex. The last column of Table 3 gives the percentage of “binders” in the ensembles. The fraction of “binders” correlates with the experimentally measured binding affinity for the series of peptides. The linear peptides are adopted by streptavidin at very low percentage (from 0.85% to 1.1%) compared with the cyclic or disulfide bonded peptides (from 7% to 28.7%). The measured binding affinity for the linear peptides is also much lower than the other peptides. This is caused by the entropy effect. The linear peptides are not constrained in conformational space and lose more entropy when they bind to the target protein. Therefore, the measured binding affinity and calculated “binder” fraction for the linear peptides is very low.

The last two peptides listed in Table 3 were selected from a phage display library. There are two disulfide bonds in each peptide. The conformation is more restricted than the peptides with one disulfide. It may be reasonable to expect an even higher affinity than the cyclic peptides because the conformation is more restricted by the two disulfide bonds. The measured binding affinity is actually less than that of some of the cyclic peptides. The modeled fraction of “binders” also behaves like the measured affinity. This may be caused by the geometry of the binding site for this system. Although the peptide is more rigid and has a higher fraction of HPQ-like conformers, the chance to collide with streptavidin is higher because the miniprotein is too large to properly fit the environment at the binding site. The penalty from the collision is even greater than the advantage from rigidity of the peptides. The number of times was counted that each residue collided with streptavidin, assuming that each atom on the peptide collides with streptividin only one time. FIG. 9 shows the number of collisions for each residue for the two disulfide bonded peptides. The second loop containing residues 7-11 (GMVEE) collides with streptavidin more often than other residues.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

U.S. Pat. No. 4,554,101

U.S. Pat. No. 5,220,007

U.S. Pat. No. 5,284,760

U.S. Pat. No. 5,354,670

U.S. Pat. No. 5,366,878

U.S. Pat. No. 5,389,514

U.S. Pat. No. 5,440,013

U.S. Pat. No. 5,446,128

U.S. Pat. No. 5,475,085

U.S. Pat. No. 5,618,914

U.S. Pat. No. 5,635,377

U.S. Pat. No. 5,670,155

U.S. Pat. No. 5,672,681

U.S. Pat. No. 5,674,976

U.S. Pat. No. 5,789,166.

U.S. Pat. No. 5,929,237

Akke, M., et al., (1993) Biochemistry 32, 9832-9844.

Alberts et al. (1994) Molecular Biology of the Cell, p 57.

Bai, Y. et al., (1995). Science, 269, 192-197.

Baldwin, R. L. (1986). Proc. Natl. Acad. Sci. USA, 83, 8069-8072.

Bolin, J. T., et al., (1982) J. Biol. Chem. 257, 13650-13662.

Bruccoleri, R. E. and Karplus, M. (1987), Biopolymers, Vol.26, 137-168.

Bystroff, C. & Kraut, J. (1991) Biochemistry 30, 2227-2239.

Bystroff, C., Oatley, S. J. & Kraut, J. (1990) Biochemistry 29, 3263-3277.

Cameron, C. E. & Benkovic, S. J. Biochemistry 36, 15792-15800.

D'Aquino, J. A., et al., (1996). Proteins: Struct. Funct. Genet. 25, 143-156.

Fierke, C. A. & Benkovic, S. J. (1989) Biochemistry 28, 478-486.

Fierke, C. A., et al., (1987) Biochemistry 26, 4085-4092.

Freire E. & Xie, D. (1994). Biophys. Chem. 51, 243-251.

Freire, E. (1995). Annu. Rev. Biophys. Biomol. Struct. 24, 141-165.

Freire, E. (1999) Proc. Natl. Acad. Sci. USA 96, 10118-10122.

Freire, E. et al., (1993). Proteins: Struct. Funct. Genet. 17, 111-123.

Gomez J. & Friere, E. (1995). J. Mol. Biol. 252, 337-350.

Gomez, J., et al., (1995). Proteins: Struct. Funct. Genet. 22, 404-412.

Griko, Y., et al., (1994). Biochemistry, 33, 1889-1899.

Griko, Y. V., et al., (1995). J. Mol. Biol. 252, 447-459.

Haynie, D. T. & Freire, E. (1993). Proteins: Struct. Funct. Genet. 16, 115-140.

Hilser, V. J. & Freire, E. (1996) J. Mol. Biol 262, 756-772.

Hilser, V. J. & Freire, E. (1997) Proteins Struct. Funct. Genet. 27, 171-183.

Hilser, V. J., et al., (1998) Proc. Natl. Acad. Sci. USA 95, 9903-9908.

Hilser, V. J., et al., (1996) Proteins 26, 123-133.

Hilser, V. J., et al., (1997) Biophys. Chem. 64, 69-79.

Iijima, H., et al., (1987) Prot: Struct, Funct, and Genet 2, 330-339

Jacobs, M. D. & Fox, R. O. (1994). Proc. Natl Acad. Sci. USA, 91, 449-453.

Jennings, P. A. & Wright, P. E. (1993). Science, 262, 892-896.

Johannesson et al., (1999) J. Med. Chem. 42:601-608.

Johnson, M. S. et al., (1993) J. Mol. Biol., 231:735-52.

Jones, B. E. & Matthews, C. R. (1995). Protein Sci. 4, 167-177.

Kyte & Doolittle (1982) J Mol Biol. 157:105-32.

Lee, K. H. et al., (1994). Proteins: Struct. Funct. Genet. 20, 68-84.

Matthews, C. R. (1993). Annu. Rev. Biochem. 62, 653-683.

Metropolis, N., et al.,(1953) J. Chem. Phys. 21, 1087-1092.

Momany, F. A., et al., (1975) J. Phys. Chem. 79, 2361-2381.

Murphy, K. P. & Freire, E. (1992) Adv. Protein Chem. 43, 313-361.

Murphy, K. P., et al., (1992). J. Mol. Biol. 227, 293-306.

Ohmae, E., et al., (1996) J. Biochem. 119, 703-710.

Ohmae, E., et al., (1998) J. Biochem. 123, 839-846.

Olejniczak, E. T., et al., (1997) Biochemistry 36, 4118-4124.

Pabo, C. 0. & Lewis, M. (1982) Nature (London) 298, 443-447.

Pace, C. N., et al., (1988). J. Biol. Chem. 263, 11820-11825.

Pedersen, T. G., et al., (1991). J. Mol. Biol 218, 413-426.

Peng, Z. & Kim, P.S. (1994). Biochemistry, 33, 2136-2141.

Ponder, J. W. & Richards, F. M. (1987), J. Mol. Biol. Vol 193, 775-791

Ramachandran, et al., (1963). J. Mol. Biol. 7, 95.

Remington's Pharmaceutical Sciences, 18th Ed. Mack Printing Company, 1990.

Schulman, B. A., et al., (1995). J. Mol. Biol. 253, 651-657.

Sosnick, T. R., et al, (1994). Nature Struct. Biol. 1, 149-156.

Stivers, J. T., et al., Biochemistry 35, 16036-16047.

Vita et al., 1998, Biopolymers 47:93-100.

Warren, M. S., et al., (1991) Biochemistry 30, 11092-11103.

Weisshoff et al., 1999, Eur. J. Biochem. 259:776-788.

Wells, J. A. (1996) Proc Natl Acad Sci USA. 93(1):1-6.

Xie, D. & Freire, E. (1994a). Proteins: Struct. Funct. Genet. 19: 291-301.

Xie, D. & Freire, E. (1994b). J. Mol. Biol. 242: 62-80.

Xie, D., Fox, R. & Freire, E. (1994). Protein Sci. 3, 2175-2184

Yu, L., et al., (1996) Biochemistry 35, 9661-9666.

Zidek, L., et al., (1999) Nat. Struct. Biol. 6, 1118-1121.

We claim:

1. A method of designing a protein pharmaceutical exhibiting optimized pharmaceutical properties comprising the steps of:
i. obtaining a test data set of variants of the protein pharmaceutical;
ii. preparing a library of ensemble derived properties for the test data set using a computer based method;
iii. obtaining experimental data for a given property for each protein variant within the test data set;
iv. deriving a parametric equation using the experimental data and the library of ensemble derived properties; and
v. creating a protein pharmaceutical with the structural characteristics found by the above steps to provide optimized pharmaceutical properties.

2. A method of designing a protein pharmaceutical exhibiting increased binding affinity between the protein pharmaceutical and a ligand comprising the steps of:
i. obtaining a test data set of variants of the protein pharmaceutical;
ii. preparing a library of ensemble derived properties for the test data set using a computer based method;
iii. obtaining experimental data for a given property for each protein variant within the test data set;
iv. deriving a parametric equation using the experimental data and the library of ensemble derived properties; and
v. creating a protein pharmaceutical with the structural characteristics found by the above steps to provide increased binding affinity between the protein pharmaceutical and a ligand.

3. The method of claim 2, wherein the binding affinity is determined by surface plasmon resonance.

4. The method of claim 2, wherein the ligand is a protein.

5. The method of claim 2, wherein determining the fraction of conformations capable of binding and the binding affinity comprises performing van der Waals calculations with the protein and the ligand to verify whether the conformation is sterically allowed.

6. The method of claim 2, wherein determining the fraction of conformations capable of binding and the binding affinity comprises determining the association or dissociation constant of the binding between the protein and the ligand.

7. The method of claim 2, further comprising determining the conformers that decrease the entropy of binding by stabilizing structures similar to that of the protein in a bound state with the ligand.

8. The method of claim 2, wherein the protein inhibits the binding of a ligand to a receptor by binding the non-protein ligand at the receptor-binding site.

9. A method of designing a protein pharmaceutical exhibiting decreased aggregation comprising the steps of:
i. obtaining a test data set of variants of the protein pharmaceutical;
ii. preparing a library of ensemble derived properties for the test data set using a computer based method;
iii. obtaining experimental data for a given property for each protein variant within the test data set;
iv. deriving a parametric equation using the experimental data and the library of ensemble derived properties; and
v. creating a protein pharmaceutical with the structural characteristics found by the above steps to provide decreased aggregation.

10. The method of claim 9, wherein aggregation is measured by light scattering at 360 nm.

11. The method of claim 9, wherein the number of hydrophobic residues exposed on the surface of the protein is reduced.

12. The method of claim 9, wherein the number of unfolded regions found in equilibrium is reduced.

13. The method of claim 9, wherein the number of glutamine/asparagine-rich domains is decreased.

14. A method of designing a protein pharmaceutical exhibiting increased solubility comprising the steps of:
i. obtaining a test data set of variants of the protein pharmaceutical;
ii. preparing a library of ensemble derived properties for the test data set using a computer based method;
iii. obtaining experimental data for a given property for each protein variant within the test data set;
iv. deriving a parametric equation using the experimental data and the library of ensemble derived properties; and
v. creating a protein pharmaceutical with the structural characteristics found by the above steps to provide increased solubility.

15. The method of claim 14, wherein solubility is measured by determining the free transfer energy of the protein pharmaceutical.

16. The method of claim 14, wherein the number of polar residues on the surface of the protein is increased.

17. The method of claim 14, wherein the number of nonpolar residues on the surface of the protein is decreased.

18. The method of claim 14, wherein the net charge of the protein is increased.

19. A method of designing a protein pharmaceutical exhibiting decreased immunogenic effects comprising the steps of:
i. obtaining a test data set of variants of the protein pharmaceutical;
ii. preparing a library of ensemble derived properties for the test data set using a computer based method;
iii. obtaining experimental data for a given property for each protein variant within the test data set;
iv. deriving a parametric equation using the experimental data and the library of ensemble derived properties; and
v. creating a protein pharmaceutical with the structural characteristics found by the above steps to provide decreased immunogenic effects.

20. The method of claim 19, wherein the immunogenic effects are determined by ELISA.

21. The method of claim 19, wherein the protein pharmaceutical exhibits a decreased tendency to aggregate with other molecules of the protein pharmaceutical.

22. The method of claim 19, wherein the protein pharmaceutical exhibits a decreased tendency to bind to the proteins of the major histocompatability complex.

23. The method of claim 19, wherein the protein pharmaceutical is a protein of less than 5,000 Daltons.

24. The method of claim 19, wherein the protein pharmaceutical is resistant to processing by the endosomal pathway.

25. A protein pharmaceutical exhibiting optimized pharmaceutical properties having structural characteristics determined the method of claim 1.

26. The protein pharmaceutical of claim 25, wherein the protein pharmaceutical is systemically or mucosally administered to a subject in a therapeutically effective amount.

27. A protein pharmaceutical exhibiting increased binding affinity between said protein and a ligand, having the structural characteristics determined by the method of claim 2.

28. A protein pharmaceutical exhibiting decreased aggregation, having the structural characteristics determined by the method of claim 9.

29. A protein pharmaceutical exhibiting increased solubility, having the structural characteristics determined by the method of claim 14.

30. A protein pharmaceutical exhibiting decreased immunogenic effects, having the structural characteristics determined by the method of claim 19.

31. A computer system for designing a protein pharmaceutical exhibiting optimized pharmaceutical properties comprising:
i. a database containing a test data set of variants of a protein pharmaceutical; and
ii. a software program coupled with said database, the software program adapted for performing the steps of:
preparing a library of ensemble derived properties for the test data set,
generating experimental data for a given property for protein variants within the test data set,
deriving a parametric equation using the experimental data and the library of ensemble derived properties, and
creating a protein pharmaceutical structure with structural characteristics found by the above steps

32. The computer system of claim 31 wherein the designed protein pharmaceutical is designed to provide optimized pharmaceutical properties.

33. The computer system of claim 31 wherein the designed protein pharmaceutical is designed to provide increased binding affinity between the protein pharmaceutical and a ligand.

34. The computer system of claim 31 wherein the designed protein pharmaceutical is designed to provide decreased immunogenic effects.

35. A computer-readable storage medium having stored therein a software program which executes the steps of:
i. preparing a library of ensemble derived properties from a test data set of variants of a protein pharmaceutical;
ii. generating experimental data for a given property for protein variant within the test data set;
iii. deriving a parametric equation using the experimental data and the library of ensemble derived properties; and
iv. creating a protein pharmaceutical structure with the structural characteristics found by the above steps.

36. The computer-readable storage medium of claim 35, wherein the designed protein pharmaceutical is designed to provide optimized pharmaceutical properties.

37. The computer-readable storage medium of claim 35, wherein the designed protein pharmaceutical is designed to provide increased binding affinity between the protein pharmaceutical and a ligand.

38. The computer-readable storage medium of claim 35, wherein the designed protein pharmaceutical is designed to provide decreased immunogenic effects.

39. A computer-implemented method for designing a protein pharmaceutical exhibiting optimized pharmaceutical properties, said method comprising:
i. preparing a library of ensemble derived properties from a test data set of variants of a protein pharmaceutical;
ii. generating experimental data for a given property for protein variants within the test data set;
iii. deriving a parametric equation using the experimental data and the library of ensemble derived properties; and
iv. creating a protein pharmaceutical structure with the structural characteristics found by the above steps.

40. The computer-implemented method of claim 39, wherein the designed protein pharmaceutical is designed to provide optimized pharmaceutical properties.

41. The computer-implemented method of claim 39, wherein the designed protein pharmaceutical is designed to provide increased binding affinity between the protein pharmaceutical and a ligand.

42. The computer-implemented method of claim 39, wherein the designed protein pharmaceutical is designed to provide decreased immunogenic effects.
